(19) United States
(12) Patent Application Publication
(10) Pub. No.: US 2023/0290434 A1
(43) Pub. Date: Sep. 14, 2023

(54) REGULARIZED DEEP LEARNING BASED IMPROVEMENT OF BIOMOLECULES

(71) Applicant: YALE UNIVERSITY, New Haven, CT (US)

(72) Inventors: Egbert Castro, New Haven, CT (US); Smita Krishnaswamy, Madison, CT (US); Abhinav Godavarthi, Plano, TX (US); Julian Rubinfien, New York, NY (US)

(21) Appl. No.: 18/061,272

(22) Filed: Dec. 2, 2022

Related U.S. Application Data
(60) Provisional application No. 63/285,647, filed on Dec. 3, 2021.
(60) Provisional application No. 63/285,783, filed on Dec. 3, 2021.

(51) Int. Cl. G16B 15/20; G16B 40/20

(52) U.S. Cl. CPC G16B 15/20; G16B 40/20

(57) ABSTRACT

A system for identifying biomolecules with a desired property comprises a computer-readable medium with instructions stored thereon, which when executed by a processor perform steps comprising collecting a quantity of biomolecular data, transforming the biomolecular data from a sequence space to a latent space representation of the data, compressing the latent space representation using a pooling mechanism, compressing the coarse representation of the biomolecular data using an informational bottleneck, calculating a fitness factor of each data element in the low-dimensional representation of the biomolecular data, choosing a point from within the low-dimensional representation of the biomolecular data, calculating a set of gradients of the fitness factor, selecting an adjacent point having the highest gradient and setting it as the first point, then repeating the gradient calculating step until the fitness factor reaches a convergence point. A method for identifying biomolecules with a desired property is also disclosed.

### CROSS-REFERENCE TO RELATED APPLICATIONS

This application claims priority to U.S. Provisional Application No. 63/285,647 filed on Dec. 3, 2021, and U.S. Provisional Application No. 63/285,783, filed on Dec. 3, 2021, both of which are incorporated herein by reference in their entirety.

### STATEMENT REGARDING FEDERALLY SPONSORED RESEARCH OR DEVELOPMENT

This invention was made with government support under GM135929 and LM007056 awarded by National Institutes of Health. The government has certain rights in the invention.

### BACKGROUND OF THE INVENTION

The primary challenge in sequence-based protein design is the vast space of possible sequences. A small protein of 30 residues (average length in eukaryotes ≈ 472) translates into total search space of 10³⁸ — far beyond the reach of modern high-throughput screening technologies. This obstacle is further exacerbated by epistasis — higher-order interactions between amino acids at distant residues in the sequence — which makes it difficult to predict the effect of small changes in the sequence on its properties. Together, this motivates the need for approaches that can better leverage sequence-function relationships, often described using fitness landscapes, to more efficiently generate protein sequences with desired properties. To address this problem, disclosed herein is a data-driven deep generative approach. The disclosed approach leverages the greater abundance of labeled data, arising from recent improvements in library generation and phenotypic screening technologies, to learn a highly structured latent space of joint sequence and structure information. Further, the disclosed system introduces novel regularizations to the latent space in the disclosed method such that molecules can be improved and redesigned directly in the latent space using gradient ascent on the fitness function.

Although the fitness (the term “fitness” is used herein to refer to some quantifiable level of functionality that a protein sequence possesses, e.g. binding affinity, fluorescence, catalysis, and stability) is more directly a consequence of the folded, three-dimensional structure of the protein rather than strictly its amino acid sequence, it is often preferable to connect fitness directly to sequence since structural information may not always be available. Indeed, when generating a library of variants for therapeutic discovery or synthetic biology, either through a designed, combinatorial approach or by random mutagenesis, it is cost-prohibitive to solve for the structure of each of the typically 10³ to 10⁹ variants produced.

Protein design is fundamentally a search problem in a complex and vast space of amino acid sequences. For most biologically relevant proteins, sequence length can range from few tens to several thousands of residues. Since each position of a N-length sequence may contain one of 20 possible amino acids, the resulting combinatorial space (≈ 20^{N} sequences) is often too large to search exhaustively. Notably this problem arises with the consideration of just canonical amino acids, notwithstanding the growing number of noncanonical alternatives. A major consequence of the scale of this search space is that most publicly available datasets, though high throughout in their scale, capture only a small fraction of the possible sequence space and thus the vast majority of possible variants are left unexplored.

To navigate the sequence space, an iterative search procedure called directed evolution (Arnold, F. H. Acc. Chem. Res. (1998)) is often applied, where a starting sequence is slowly mutated, one amino acid or base at a time. The best sequence or sequences are then carried over to the next round of library generation and selection. Effectively, this searches sequence space using a hill climbing approach and as a consequence, is susceptible to local maxima that may obscure the discovery of better sequences. This method of exploration is also very slow in that it hovers around the region of the starting sequence for long periods of time. Other approaches to protein design include structure-based design (Rohl, C. A., et al., Methods Enzymol. (2004); Norn, C. et al., Proc. Natl Acad. Sci. USA (2021)), where ideal structures are chosen a priori and the task is to fit a sequence to the design. Recently, several promising approaches have emerged incorporating deep learning into the design (Brookes, D. H. (2018); Brookes, D., Proceedings of the 36th International Conference on Machine Learning (2019)), search (Yang, K. K., et al., Nat. Methods (2019); Biswas, S., et al, Nat. Methods (2021)), and optimization (Linder, J. (2020)). of proteins. However, these methods are typically used for in-silico screening by training a model to predict fitness scores directly from the input amino acid sequences. Recent approaches have also utilized reinforcement learning to improve sequences (Angermueller, C. et al. International Conference on Learning Representations (2019)). Although these approaches are valuable for reducing the experimental screening burden by proposing promising sequences, the challenge of navigating the sequence space remains unaddressed.

### SUMMARY OF THE INVENTION

In one aspect, a system for identifying biomolecules with a desired property comprises a non-transitory computer-readable medium with instructions stored thereon, which when executed by a processor perform steps comprising collecting a quantity of biomolecular data, transforming the biomolecular data from a sequence space to a latent space representation of the biomolecular data, compressing the latent space representation of the biomolecular data to a coarse representation using a pooling mechanism, compressing the coarse representation of the biomolecular data to a low-dimensional representation of the biomolecular data using an informational bottleneck, organizing the data in the low-dimensional representation of the biomolecular data according to a fitness factor, choosing a first point from within the low-dimensional representation of the biomolecular data, calculating a gradient of the fitness factor at the first point in the low-dimensional representation of the biomolecular data, selecting a second point in the low-dimensional representation of the biomolecular data in a direction indicated by the gradient to have a higher fitness factor than the first point, setting the second point as the first point, then repeating the gradient calculating step until the fitness factor reaches a convergence point or threshold value, and transforming the selected point from within the low-dimensional representation of the biomolecular data back to the sequence space to identify an improved candidate sequence.

In one embodiment, the pooling mechanism is an attention-based pooling mechanism. In one embodiment, the pooling mechanism is a mean or max pooling mechanism. In one embodiment, the pooling mechanism is a recurrent pooling mechanism. In one embodiment, the informational bottleneck is an autoencoder-type bottleneck. In one embodiment, the instructions further comprise the step of adding negative samples to the latent space representation of the biomolecular data. In one embodiment, the negative samples have a fitness value less than or equal to the minimum fitness value calculated in the latent space. In one embodiment, the biomolecular data comprises sequencing data of at least one lead biomolecule. In one embodiment, the instructions comprise transforming the biomolecular data to a latent space representation of the biomolecular data with a transformer module having at least eight layers with four heads per layer.

In one aspect, a method of identifying biomolecules with a desired property comprises collecting a quantity of biomolecular data, transforming the biomolecular data from a sequence space to a latent space representation of the biomolecular data, compressing the latent space representation of the biomolecular data to a coarse representation using a pooling mechanism, compressing the coarse representation of the biomolecular data to a low-dimensional representation of the biomolecular data using an informational bottleneck, organizing the data in the low-dimensional representation of the biomolecular data according to a fitness factor, choosing a first point from within the low-dimensional representation of the biomolecular data, calculating a gradient of the fitness factor at the first point in the low-dimensional representation of the biomolecular data, selecting a second point in the low-dimensional representation of the biomolecular data in a direction indicated by the gradient to have a higher fitness factor than the first point, setting the second point as the first point, then repeating the gradient calculating step until the fitness factor reaches a convergence point or threshold value, and transforming the selected point from within the low-dimensional representation of the biomolecular data back to the sequence space to identify an improved candidate sequence.

In one embodiment, the pooling mechanism is an attention-based pooling mechanism. In one embodiment, the pooling mechanism is a mean or max pooling mechanism. In one embodiment, the pooling mechanism is a recurrent pooling mechanism. In one embodiment, the informational bottleneck is an autoencoder-type bottleneck. In one embodiment, the instructions further comprise the step of adding negative samples to the latent space representation of the biomolecular data. In one embodiment, the negative samples have a fitness value have a fitness value less than or equal to the minimum fitness value calculated in the latent space.

In one embodiment, the biomolecular data comprises sequencing data of at least one lead biomolecule. In one embodiment, the instructions comprise transforming the biomolecular data to a latent space representation of the biomolecular data with a transformer module having at least eight layers with four heads per layer. In one embodiment, the method further comprises producing a protein with the improved candidate sequence.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description of various embodiments of the invention will be better understood when read in conjunction with the appended drawings. For the purpose of illustrating the invention, there are shown in the drawings illustrative embodiments. It should be understood, however, that the invention is not limited to the precise arrangements and instrumentalities of the embodiments shown in the drawings.

FIG. 1A provides a graphical view of the platform for producing the next generation of antibody therapeutics with unprecedented efficacy.

FIG. 1B provides a graphical view of the integration of latent space improvement methods.

FIG. 2 is a diagram of an exemplary computer system.

FIG. 3A shows a diagram of a method of the disclosure.

FIG. 3B shows exemplary fitness graphs of a latent space.

FIG. 3C shows exemplary diagrams of monotonic and pseudo-concave fitness functions.

FIG. 3D is a diagram of a negative sampling method as disclosed herein.

FIG. 3E and FIG. 3F are graphs of change in fitness and sequence along different walks of a fitness function.

FIG. 4A shows a comparison of different representation methods for protein sequences.

FIG. 4B shows a diagram of a method of measuring neighborhood-level variation in a latent space.

FIG. 4C, FIG. 4D, and FIG. 4E are graphs of ruggedness of fitness and sequence of different transformation methods as disclosed herein.

FIG. 5A is a graph of the quantity of high fitness sequences found in various datasets using different methods disclosed herein.

FIG. 5B is a graph of average fitness score of sequences found using different improvement methods.

FIG. 5C is a set of graphs of different fitness scores found using different improvement methods disclosed herein.

FIG. 6 is a graphical representation of the difference between searching by iterative sequence modification as compared to a gradient-based latent space search towards a property of interest.

FIG. 7 provides an overview of the use of the method for improving antibody binding affinity.

FIG. 8A is a diagram of ranibizumab and anti-ranibizumab.

FIG. 8B is a set of graphs visualizing the tight coupling in Euclidean distance in latent space, sequence distance, and fitness.

FIG. 8C is a set of graphical representations of different improvement methods.

FIG. 8D is a graphical representation of improvement paths taken by a gradient ascent method as disclosed herein.

FIG. 8E is a graphical representation of different paths taken by various gradient-free methods.

FIG. 9A is a set of visualizations of latent embeddings produced by different methods disclosed herein.

FIG. 9B is a graph of a learned fitness function extracted from a fitness prediction network disclosed herein.

FIG. 9C is a graph of a bimodal distribution of log fluorescence values with low and high fluorescence mode.

FIG. 9D is a graph of paths taken by different improvement algorithms as disclosed herein.

FIG. 10A is a diagram of a transformer function as disclosed herein.

FIG. 10B is a set of visualizations of learned pairwise relationships defined by attention maps of a transformer encoder as disclosed herein.

FIG. 10C is a diagram of an attention-based pooling step of a method as disclosed herein.

FIG. 11 is a visualization of amino acid embeddings from a method disclosed herein trained using the GIFFORD dataset comparing the AE and JT-AE models.

FIG. 12 is a visualization of amino acid embeddings from a method disclosed herein trained using the GFP dataset comparing the AE and JT-AE models.

### DETAILED DESCRIPTION

It is to be understood that the figures and descriptions of the present invention have been simplified to illustrate elements that are relevant for a clear understanding of the present invention, while eliminating, for the purpose of clarity, many other elements found in related systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

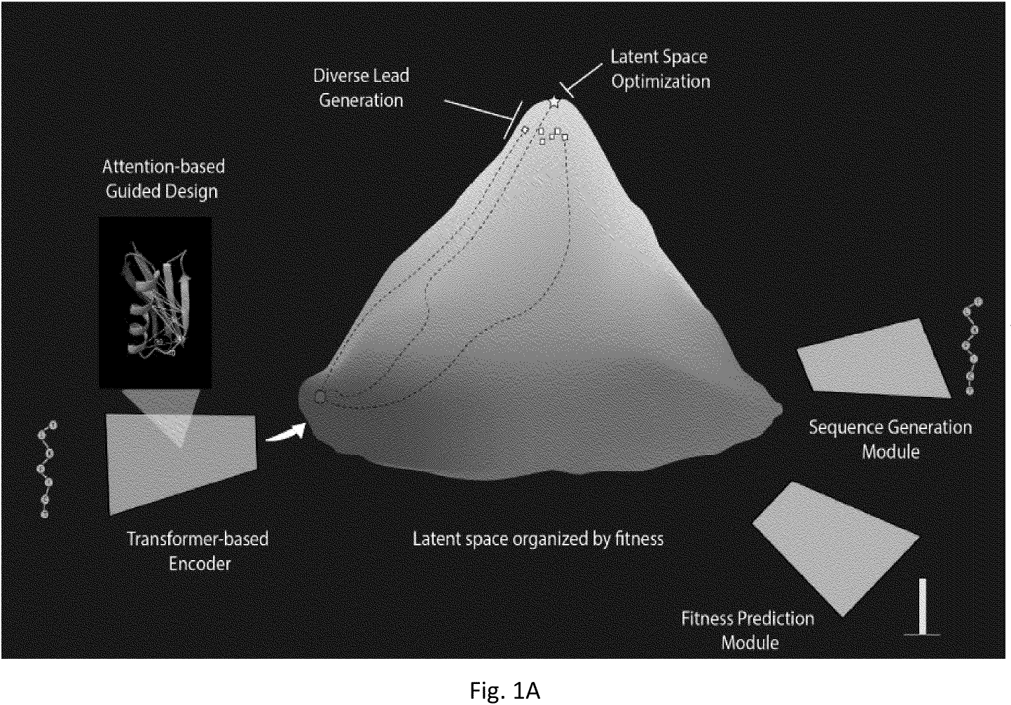

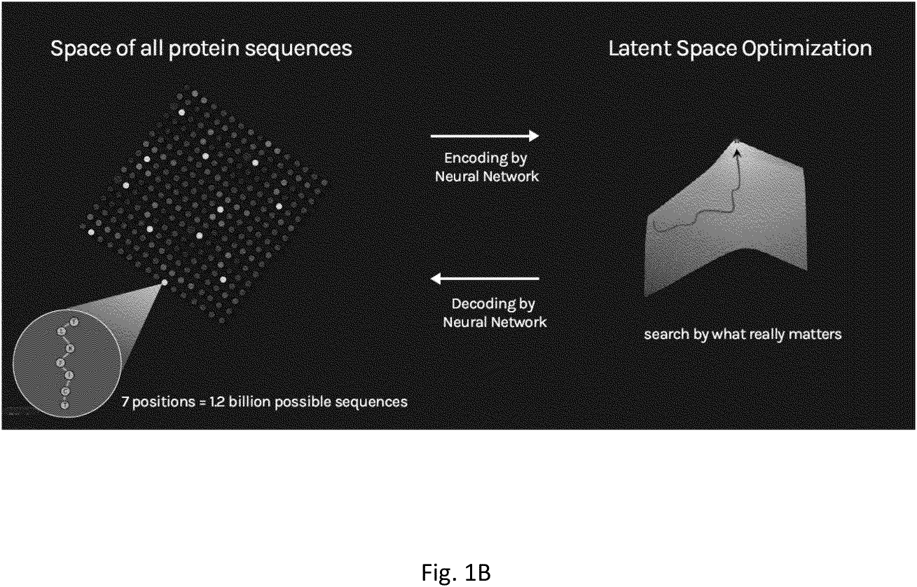

Systems and methods disclosed herein relate to improved methods for deep learning-based design and improvement of biomolecules. In one embodiment, the systems and methods disclosed herein relate to a deep learning model or neural network, for example a neural network that incorporates a latent space improvement method as depicted in FIG. 1A and FIG. 1B.

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

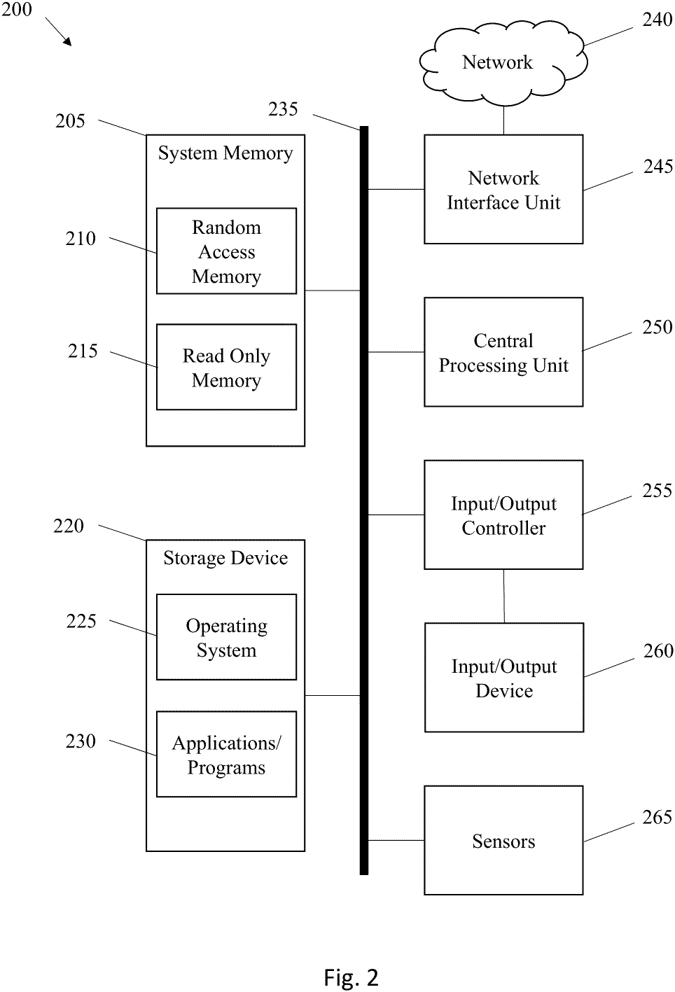

FIG. 2 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 2 depicts an illustrative computer architecture for a computer 200 for practicing the various embodiments of the invention. The computer architecture shown in FIG. 2 illustrates a conventional personal computer, including a central processing unit 250 (“CPU”), a system memory 205, including a random access memory 210 (“RAM”) and a read-only memory (“ROM”) 215, and a system bus 235 that couples the system memory 205 to the CPU 250. A basic input/output system containing the basic routines that help to transfer information between elements within the computer, such as during startup, is stored in the ROM 215. The computer 200 further includes a storage device 220 for storing an operating system 225, application/program 230, and data.

The storage device 220 is connected to the CPU 250 through a storage controller (not shown) connected to the bus 235. The storage device 220 and its associated computer-readable media provide non-volatile storage for the computer 200. Although the description of computer-readable media contained herein refers to a storage device, such as a hard disk or CD-ROM drive, it should be appreciated by those skilled in the art that computer-readable media can be any available media that can be accessed by the computer 200.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 200 may operate in a networked environment using logical connections to remote computers through a network 240, such as TCP/IP network such as the Internet or an intranet. The computer 200 may connect to the network 240 through a network interface unit 245 connected to the bus 235. It should be appreciated that the network interface unit 245 may also be utilized to connect to other types of networks and remote computer systems.

The computer 200 may also include an input/output controller 255 for receiving and processing input from a number of input/output devices 260, including a keyboard, a mouse, a touchscreen, a camera, a microphone, a controller, a joystick, or other type of input device. Similarly, the input/output controller 255 may provide output to a display screen, a printer, a speaker, or other type of output device. The computer 200 can connect to the input/output device 260 via a wired connection including, but not limited to, fiber optic, Ethernet, or copper wire or wireless means including, but not limited to, Wi-Fi, Bluetooth, Near-Field Communication (NFC), infrared, or other suitable wired or wireless connections.

As mentioned briefly above, a number of program modules and data files may be stored in the storage device 220 and/or RAM 210 of the computer 200, including an operating system 225 suitable for controlling the operation of a networked computer. The storage device 220 and RAM 210 may also store one or more applications/programs 230. In particular, the storage device 220 and RAM 210 may store an application/program 230 for providing a variety of functionalities to a user. For instance, the application/program 230 may comprise many types of programs such as a word processing application, a spreadsheet application, a desktop publishing application, a database application, a gaming application, internet browsing application, electronic mail application, messaging application, and the like. According to an embodiment of the present invention, the application/program 230 comprises a multiple functionality software application for providing word processing functionality, slide presentation functionality, spreadsheet functionality, database functionality and the like.

The computer 200 in some embodiments can include a variety of sensors 265 for monitoring the environment surrounding and the environment internal to the computer 200. These sensors 265 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

Disclosed herein is an alternative to working in the sequence space, specifically directed to a method of learning a low dimensional, semantically-rich representation of peptides and proteins. These latent representations collectively form the “latent space”, which is easier to navigate. With this approach, a therapeutic candidate can be improved using its latent representation.

One aspect of the present disclosure is a deep transformer-based approach to protein design, which combines the powerful encoding ability of a transformer model with a bottleneck that produces information-rich, low dimensional latent representations. The latent space disclosed herein, besides being low dimensional is regularized to be 1) smooth with respect to structure and fitness by way of fitness prediction from the latent space, 2) regularized to be continuous and interpolatable between training data points, and 3) pseudo-convex based on negative sampling outside the data. This latent space enables calculation of improvements directly in latent space using gradient ascent on the fitness and converges to a maximum point that can then be decoded back into the sequence space.

Key contributions of the disclosed method include:
(1) The novel use of a transformer-based encoder with an autoencoder-type bottleneck for rich and interpretable encodings of protein sequences
(2) A latent space that is organized by sequence-function relationships, which ameliorates difficulties arising from combinatorial explosion.
(3) A convex latent space that is reshaped using norm-based negative sampling to induce a natural boundary and stopping criterion for gradient-based evaluation.
(4) An interpolation-based regularization which enforces gradual changes in decoded sequence space when traversing though latent space. This allows for a more dense sampling of the underlying sequence manifold on which the training data lies.
(5) A gradient ascent algorithm for generating new sequences from the latent space.

The disclosed method is evaluated on several publicly-available protein datasets, including variant sets of anti-ranibizumab and GFP. This domain is viewed first through a protein representation learning perspective, where popular representations of proteins are compared. It was observed that the disclosed method learns a more organized, smoother representation relative to other approaches. Next the disclosed method is evaluated on several protein design tasks. Compared to other sequence-based approaches, the disclosed method shows greater efficiency (increase in fitness per step) using its fitness-directed traversal of latent space. This efficiency allows the disclosed method to more robustly generate high-fitness sequences. Lastly, the attention-based relationships learned by the jointly-trained models provide a potential avenue towards sequence-level fitness attribution information.

The disclosed architecture is designed to jointly generate protein sequences as well as predict fitness from latent representations. In one embodiment, the model is trained using a multi-task loss formulation which organizes the latent space by structure (input sequence) and function simultaneously, thus simplifying the task of finding sequences of high fitness from a search problem in a high-dimensional, discrete space to a much more amenable improvement problem in a low dimensional, continuous space. As used herein, “function” could refer to any desirable or undesirable feature of a sequence, including but not limited to toxicity, binding affinity, stability, activity, half-life, a fluorescent property, immunogenicity, energy, lipophilicity, molecular weight, sensitivity to photobleaching, drug likeness, and/or variant number of a viral protein.

In some embodiments, the method leverages a transformer encoder to learn the mapping of sequences to latent space and utilizes gradient-based methods to systematically and efficiently move through latent space towards regions of high fitness. A norm-based negative sampling penalty may be used in some embodiments to reshape the latent fitness landscape to be pseudo-convex. This has the dual benefit of further easing the improvement challenge as well as creating an implicit trust boundary. The disclosed method makes innovative use of an interpolation regularization that enforces smoothness with respect to sequence, whereby small perturbations to a latent representation correspond to minor changes in the reconstructed sequence. This is especially relevant to protein design as it allows for a dense sampling of the latent space for diverse protein sequence generation while retaining properties of interest.

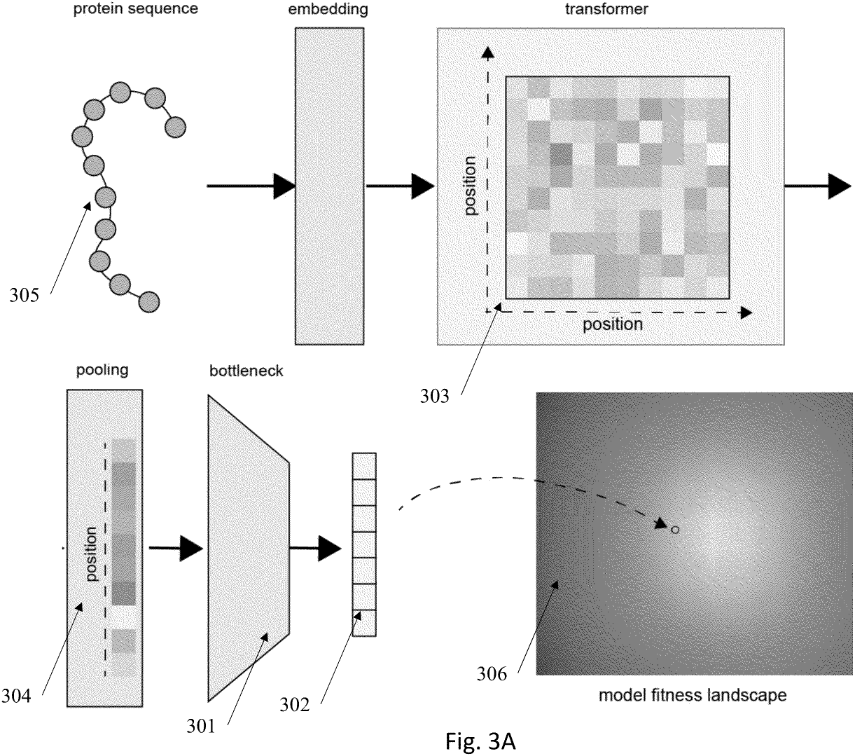

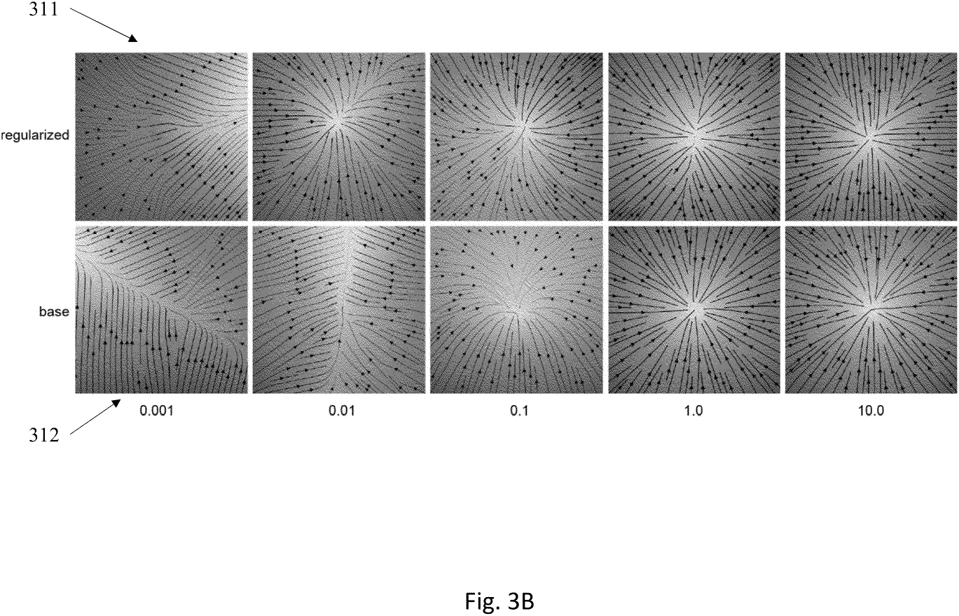

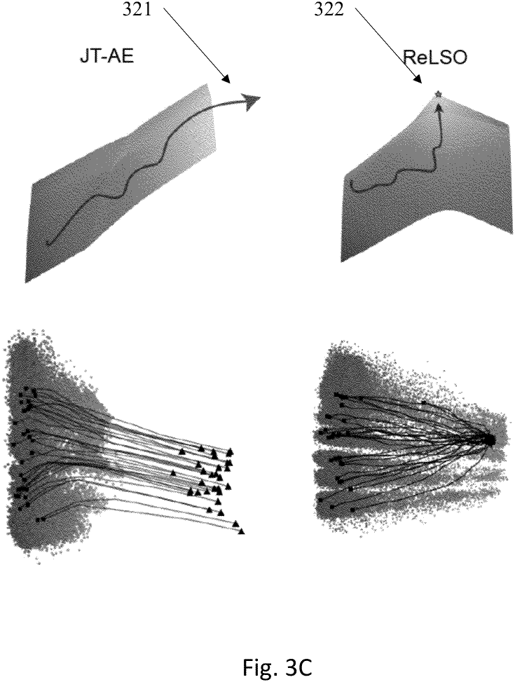

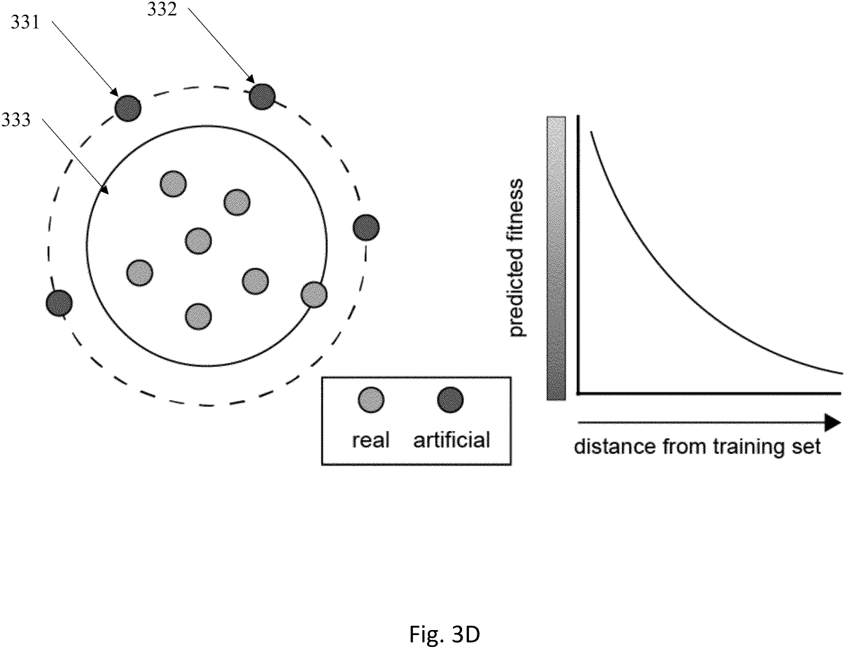

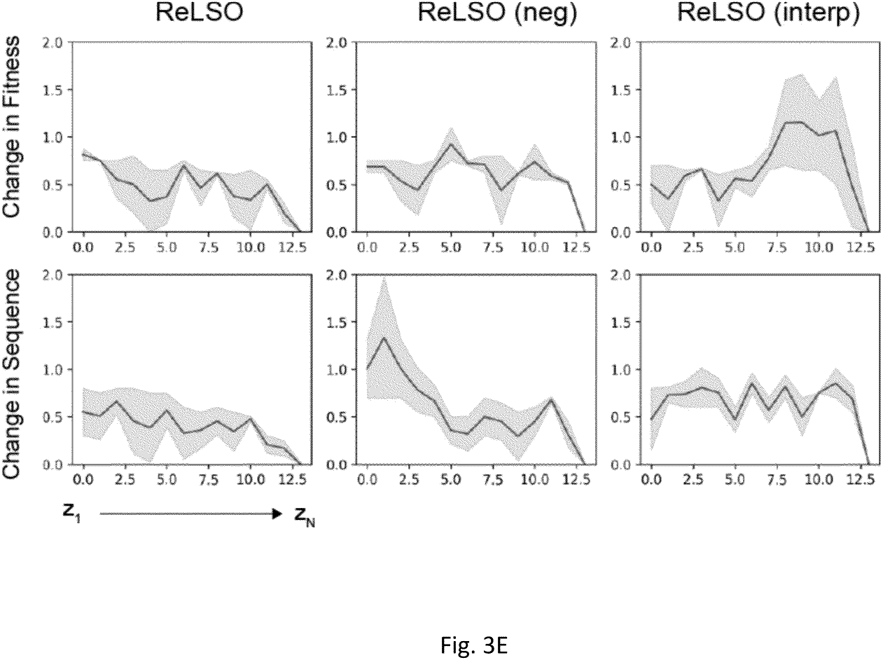

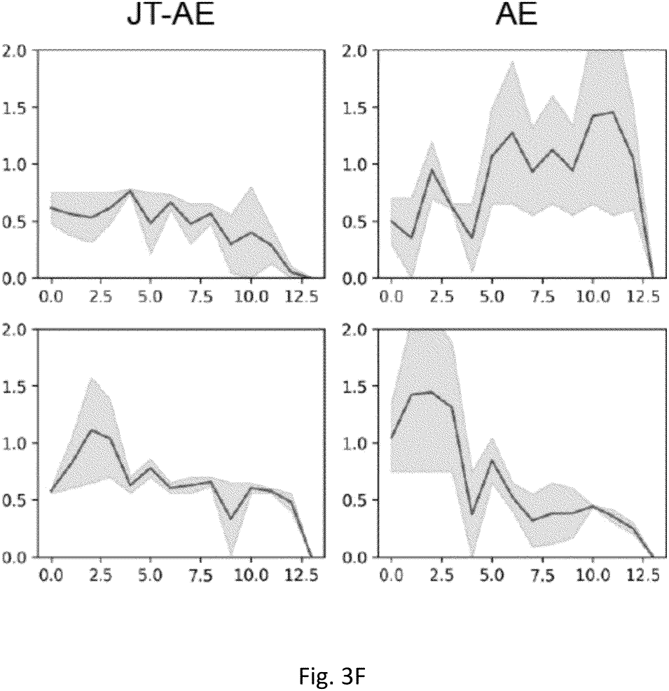

The disclosed method employs a transformer-based encoder to learn the mapping from a sequence, x, to its latent representation, z (see e.g. FIG. 3A). While other encoding methods that rely on convolutional and recurrent architectures have demonstrated success in this domain, the present method uses a transformer for several key reasons. First, the inductive bias of the transformer architecture matches the prevailing understanding that protein function is a consequence of pairwise interactions between residues (e.g. catalytic triads of proteases). Indeed the transformer architecture has shown promise in several tasks relying on protein sequence for prediction (see Jumper, J. et al. Nature (2021); Rao, R. et al. Adv. Neural Inf. Process. Syst. (2019); Rao, R., et al., (2020); Rives, A., et al. Proc. Natl Acad. Sci. USA (2021)). Secondly, the transformer’s attention-based encoding scheme provides for interpretability by analysis of learned attention weights. Finally, transformers have demonstrated advantages in representing long sequences, as a consequence of viewing the entire sequence during the forward pass, thus avoiding the limitations inherent to recurrent neural network-based encoders. A more in-depth discussion of protein sequence representation learning approaches may be found in Detlefsen, N., et al, Nat. Commun. (2022), incorporated herein by reference.

The encoder network disclosed herein, fθ, transforms input proteins to a token-level representation where each amino acid in the sequence is replaced by a positional encoding of fixed length. This representation is then compressed to a coarse, sequence-level, representation using an attention-based pooling mechanism which computes the convex sum of positional encodings. This approach is preferred over mean or max pooling since it is able to weight the importance of positions in the sequence without incurring the computational cost of more complex strategies using recurrent-based pooling. Different from other transformer encoders, in the methods disclosed herein, the dimensionality of this sequence-level representation is further reduced using a fully-connected network (see FIG. 3A). This amounts to passing the sequence information through an information bottleneck 301, resulting in an informative and low-dimensional latent representation, z (302). Low dimensionality of the latent representation is important, since latent space grows exponentially with increasing dimension making latent space improvement methods more challenging.

The present disclosure incorporates two vital factors in protein design: 1) sequence and 2) fitness information. By jointly training an autoencoder with a prediction network, the original autoencoder architecture, comprised of an encoder f_{θ} and decoder g_{θ}, is supplemented with a network h_{θ} which is tasked with predicting fitness from the latent representation z. The resulting objective function of this set-up takes the form

$$L = \left|\left|{\text{g}}_{θ} \left({\text{f}}_{θ} \left(\text{x}\right)\right) - \text{x}\right|\right| + \left|\left|{\text{h}}_{θ} \left({\text{f}}_{θ} \left(\text{x}\right)\right) - \text{y}\right|\right| \qquad \text{Equation 1}$$

which includes the reconstruction loss and the fitness prediction (regression) loss. At each backpropagation step, the encoder is updated with gradients from both losses and is therefore directed to encode information about sequence and fitness in the latent representation, z. Indeed, when the dimension of z is set to some low value, d « N, the encoder is forced to include only the most salient information about sequence and fitness and induces a connection between the two in z. This property was first exploited in the biomolecular domain (see Gomez-Bombarelli, R. et al. ACS Cent. Sci. (2018)) where a j ointly-trained variational autoencoder generated latent encodings organized by chemical properties. The disclosed method leverages the same strategy to establish a strong correspondence between the protein sequence and its fitness, which is later utilized for generating novel sequences with desired fitness. Note that the model architecture trained with the reconstruction and fitness prediction losses is referred to herein as “JT-AE” (jointly trained autoencoder).

With reference to FIG. 3A, To encode protein sequences, one embodiment of the disclosed method uses a transformer-based encoder. The output of the transformer module 303 is pooled using an attention-based pooling mechanism 304 and then compressed further, for example using a bottleneck 301 to produce the latent representation 302 of an input sequence 305. The collection of latent points forms a model fitness landscape 306. As shown in FIG. 3B, one embodiment of the disclosed method uses an auxiliary network to predict fitness from latent space and uses a norm-based negative sampling technique to enforce a pseudo-concave shape in the resulting latent space. The choice of the auxiliary network affects how sensitive the network is to the negative sampling loss. The plots in the top row 311 were generated using a network penalized to learn smoother functions whereas the plots in the bottom row 312 were produced by a conventional fully-connected network.

Although certain examples are presented herein using a transformer-based encoder as the transformer module 303 in FIG. 3A, it is understood that in other embodiments, a molecular graph may be used as the input 305 (a sequence in FIG. 3A) and where a molecular graph is used as an input, a graph neural network could be used in place of transformer 303. More information about graph neural networks in such an embodiment may be found in Castro, E., et al., 2020 IEEE International Conference on Big Data (2020), incorporated herein by reference.

Similarly, although some examples may be presented herein which describe a protein-encoding sequence as the input of a method disclosed herein, it is understood that any biomolecular data may be used as the input, including but not limited to nucleotide sequences, Simplified molecular-input line-entry system (SMILES) strings, or the like.

With reference to FIG. 3C, an inherent weakness of a naive joint-training approach, as in JT-AE, is that often a monotonic function is learned by the auxiliary network. Such a function lacks any stopping criterion when used for latent space improvement methods (see 321). To address this, in some embodiments the fitness function is reshaped such that the global maxima 322 lie in/near the training data. With reference to FIG. 3D, negative sampling relies on a data augmentation strategy wherein artificial low-fitness points (e.g. 331, 332) are generated on the periphery of latent space 333. With reference to FIG. 3E, changes in sequence and fitness were monitored during latent space traversal using 100 sampled walks between pairs of distant points in latent space. The x-axis denotes step index along the path taken. The y-axis displays the difference in the denoted property between the current step, Zᵢ, and the final step, _{Zn}, such that the difference becomes zero at the final step when Zᵢ =zₙ. The mean (line) and 95th percentile confidence interval (shaded region) are shown in the graphs of FIG. 3E and FIG. 3F.

A fundamental challenge in performing improvements in the latent space is that the improvement trajectory can stray far from the training data into regions where the prediction accuracy of the model deteriorates, producing untrustworthy results. Recent work has proposed techniques to define boundaries for model-based improvements by imposing constraints like a sequence mutation radius, or by relying on model-derived likelihood values. While mutation radii are straightforward to implement, the significant variability of fitness levels even within the immediate mutational neighborhood of a protein sequence makes such global thresholds less than ideal. Additionally, a mutational radii constraint can be oversimplified as high mutation count may potentiate functional benefit to the fitness of a protein (e.g., the B. 1.1.529 Omicron Spike protein).

The fitness prediction head of the JT-AE disclosed herein provides directional information for latent space improvements. However, it does not impose any stopping criterion nor any strong notion of a boundary or fitness optima. Furthermore, the addition of an auxiliary attribute prediction task, e.g. fitness prediction, to an autoencoder often results in unidirectional organization of the latent space by that attribute. In such cases (e.g. FIG. 3C), following the gradient produces an improvement trajectory that extends far outside the training manifold with no natural stopping point. This may ultimately result in generated protein sequences that are ‘unrealistic’ with respect to other biochemical properties necessary for proper folding such as stretches of homopolymers or amino acid sequence motifs known to be deleterious to stability/solubility in solution.

In order to fully leverage the gradient signal provided by the fitness prediction head, h_{θ}, a bias is introduced in the learned fitness function, ϕ_{z}, towards regions in the latent space near the training data. This is done using a data augmentation technique called norm-based negative sampling. Each latent representation, z, obtained from the training data is complemented with a set of negative samples, zₙ. These negative examples are produced by sampling high-norm regions of the latent space surrounding real latent points (see FIG. 3D). By assigning these artificial points, zₙ, low fitness (for example, a value at or below the minimum fitness score calculated in the training data) and including them in the fitness prediction loss, the learned fitness function, ϕ_{z}, is reshaped in a manner where there is a single fitness maximum located in or near the training data manifold. Using this regularization, an implicit trust region is formed, thus providing a natural stopping criterion for latent space improvements. To better examine this phenomenon, ReLSO was trained using a latent encoding dimension of two, z _{E} II82 and visualize the resulting, learned fitness function by h_{θ}. It was observed that the negative sampling regularization induces a pseudo-concave shape wherein high fitness latent points reside at the center of this 2-dimensional latent space (see FIG. 3B). Increasing the strength of this regularization using a hyperparameter n further enforces this organization. The JT-AE model augmented with this regularization is referred to herein (e.g. in FIG. 3E) as “ReLSO (neg)”.

To further improve the latent space of the disclosed jointly-trained for protein sequence improvements, a penalty is introduced which enforces smoother interpolation in latent space with respect to sequence modifications. This is appealing for sequence-based protein design as it is desirable to be able to more densely sample latent space for both analysis of latent space improvement trajectories as well as enrichment of the areas of sequence space around high fitness sequences.

Gradual changes in the decoded sequence space are enforced during latent space traversal by the addition of an interpolation regularization term. In this term, a subset of the batch of latent points is used to compute a k-nearest-neighbor (KNN) graph using pairwise Euclidean distances. A set of new latent points zₚ are then generated by interpolating between nearest neighbors. This new set of points zₚ are passed through the decoder network g_{θ} to produce a set of decoded sequences x̂ₚ. The distance between two sequences in x and their interpolant x̂ₚ is then penalized. Formally, this penalty is calculated element-wise by:

$${L}_{i n t e r p} = max \left(0 , \frac{\left|\left|{\hat{x}}_{1} - {\hat{x}}_{i}\right|\right| + \left|\left|{\hat{x}}_{2} - {\hat{x}}_{i}\right|\right|}{2} - \left|\left|{\hat{x}}_{1} - {\hat{x}}_{2}\right|\right|\right) \qquad \text{Equation 2}$$

where x̂₁ and x̂₂ are nearest neighbors in latent space and x̂i is the decoded sequence of the interpolated latent point. The JT-AE model augmented with only this regularization is referred to herein (e.g. in FIG. 3E) as ReLSO(interp). Finally the full model disclosed herein, with both negative sampling and interpolative sampling regularization, is referred to as ReLSO.

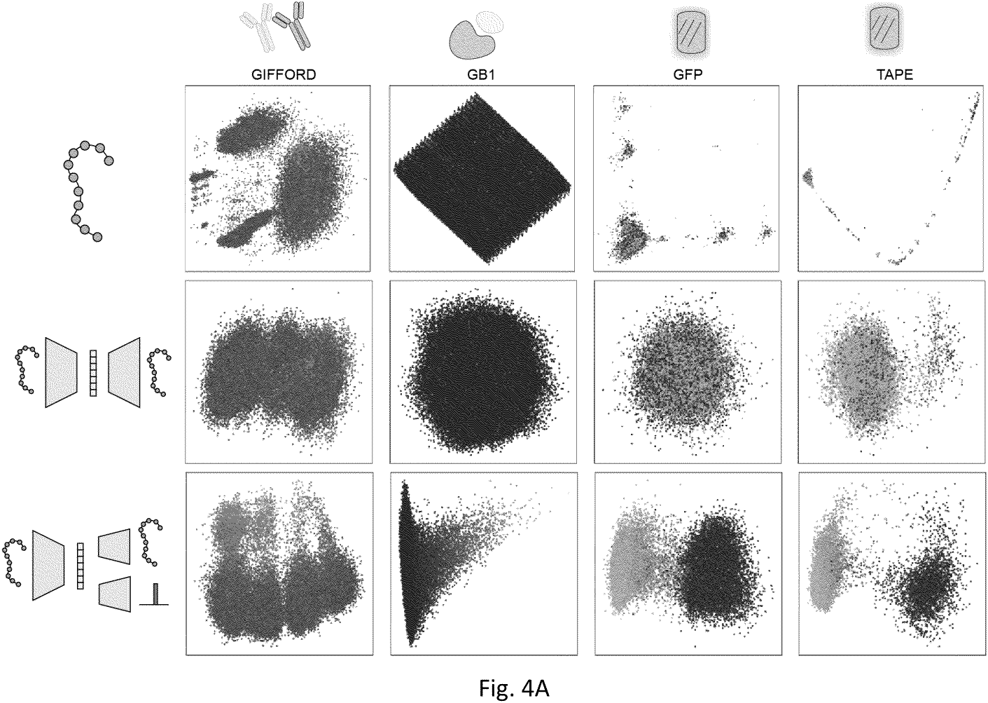

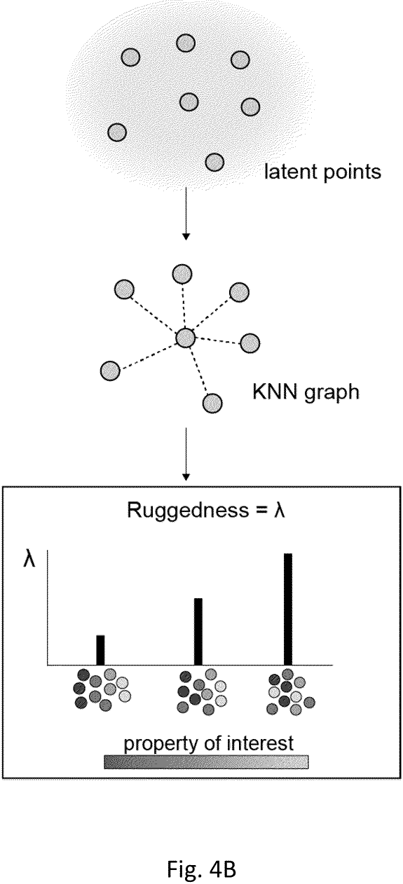

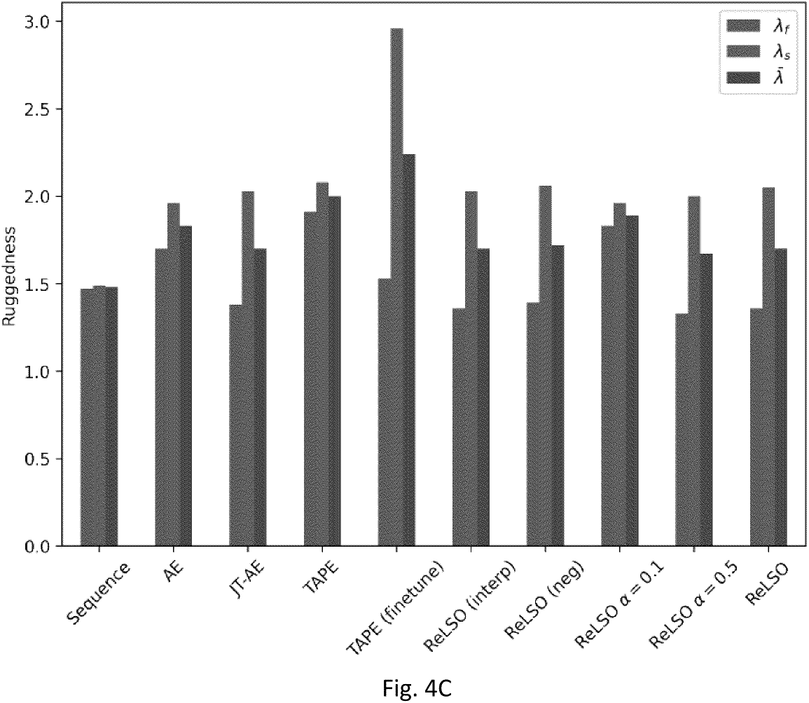

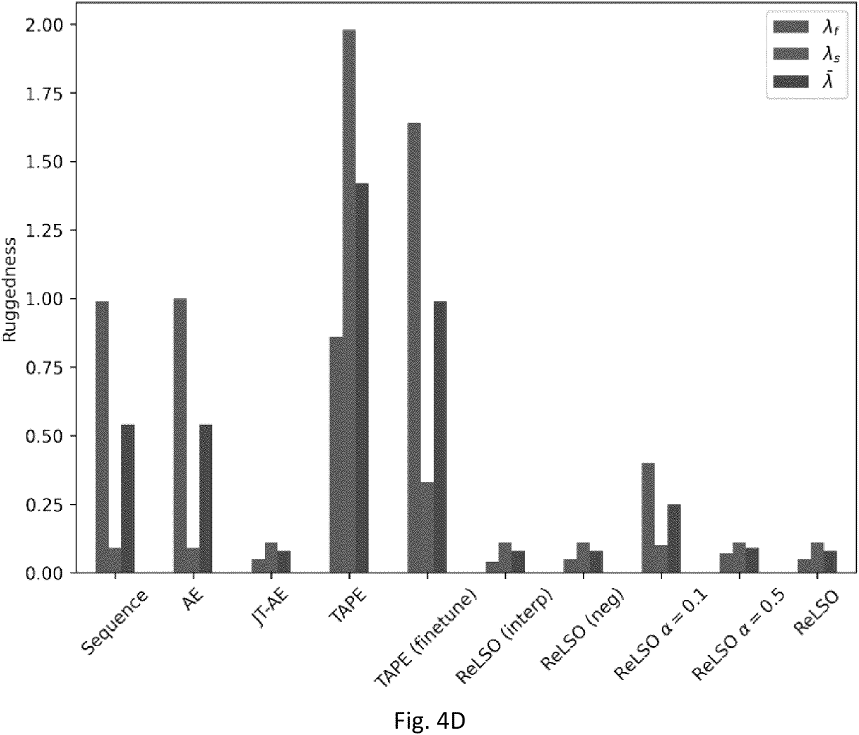

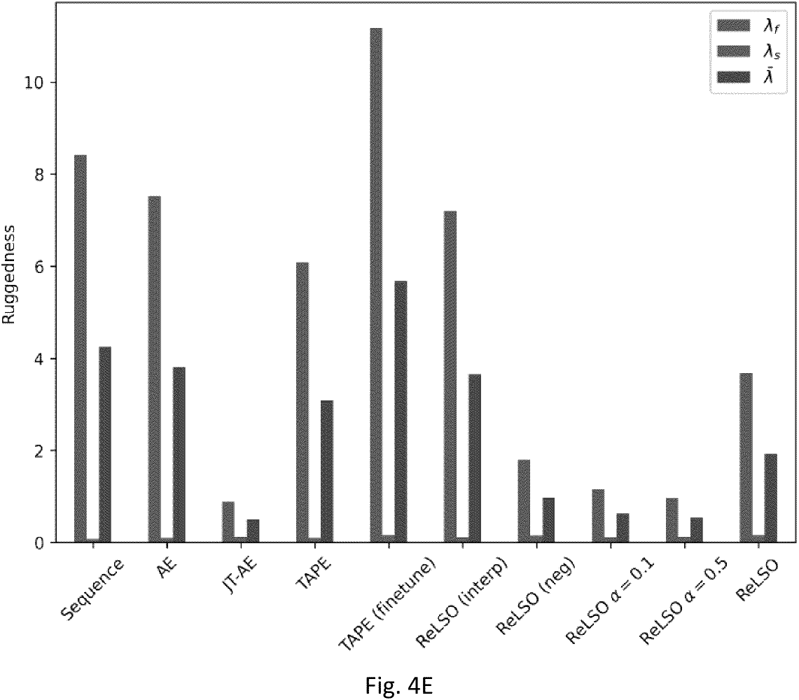

The highly-structured latent space of ReLSO is used to calculate protein sequence improvements on several publicly available datasets, with additional information on each in Table 1 below. First, the latent space maintains a global organization not only to fitness (see FIG. 4A) but to sequence information (see FIG. 4C). Next, the negative sampling and interpolative sampling regularizations induce a latent space with several properties that ease the protein sequence improvement task such as a pseudo-concave fitness function. Finally, traversal in the latent space of ReLSO results in gradual changes in both sequence and fitness (see FIG. 3E and FIG. 3F). This smoothness is quantified through the use of a graph-based metric (see FIG. 4B) derived from the graph signaling processing literature and previously used in the biomolecular domain to study thermodynamic landscapes (see Castro, E., et al., 2020 IEEE International Conference on Big Data. (2020)). Combined, these properties form a search space amenable to improvement methods.

TABLE 1

| Dataset | Sequence Length | Number of Datapoints | Diversity | Test Size |
|---|---|---|---|---|
| GB1 | 56 | 149361 | 0.068 | 0.25 |
| Gifford | 20 | 90459 | 0.695 | 0.25 |
| GFP | 237 | 54024 | 0.032 | 0.30 |
| TAPE | 237 | 54024 | 0.032 | 0.56 |

To improve protein sequences, gradient-ascent was used which allows for systematic and efficient modulation of fitness. First, a protein sequence x is encoded by the encoder network, f_{θ} to produce a latent representation z. This process maps an input protein sequence to its point in the model’s latent fitness landscape. Next, the gradient with respect to the predicted fitness for the latent point,,h_{θ}, is calculated. The determined gradient provides directional information towards the latent fitness maxima and is used to update the latent point.

$${z}^{\left(t + 1\right)} ← {z}^{\left(t\right)} + ε {∇}_{z} {h}_{θ} \qquad \text{Equation 3}$$

This iterative process requires two hyperparameters, step size (_{E}), and number of steps, K. At termination of the loop, a final latent point z_{f} is produced. This point in latent space is then decoded to its corresponding sequence xf using the decoder module ge. The reliance of a gradient signal over other, more stochastic approaches allows for a more robust approach to sequence improvement that is less sensitive to starting points. If greater sequence diversity is desired, in some embodiments, injecting noise into the update step can increase the variation in sequences produced. Overall, this process is referred to as latent space improvement, whereby protein sequences are improved in the latent space of a model rather than directly. A major benefit of using such an approach is the ability to learn a search space that ameliorates some of the challenges of improving protein sequences directly. Disclosed herein is a better search space for protein sequence improvement by heavily regularizing an autoencoder-like model such that the latent space maintains favorable properties while still being generative.

In recent years, many protein sequence improvement methods have emerged that rely on the use of a deep learning model. Some of these approaches use the model to perform an in-silico screen of candidate sequences produced by iterative or random search. These methods, however, are sequence-based search strategies, as the generation of new sequence candidates occurs at the sequence-level. In contrast, methods such as Brookes, D. H. (2018); and Brookes, D., Proceedings of the 36th International Conference on Machine Learning (2019), generate new sequences by sampling the latent space of a generative model. The methods disclosed herein seek to leverage the gradient information present in h_{θ} to search for more fit protein sequences. As a result of training to predict fitness from latent representation, it is observed that the latent space organizes by fitness, as shown in FIG. 4A. To avoid several pathologies of improvement by gradient ascent in latent space, the regularizations included in the disclosed methods reshape this organization into a pseudo-concave shape (see FIG. 3D), which eases the improvement challenge significantly.

With reference to FIG. 4A, Three main representation methods for proteins sequences are compared, namely amino acid sequence, latent embeddings from an unsupervised autoencoder, and latent embeddings produced by a jointly-trained autoencoder. The three protein sequence representation approaches are visualized using principle component analysis and each point is colored by its corresponding fitness value. Columns of the plot grid correspond to datasets referenced in Table 1 (left to right: GIFFORD, GB1, GFP, TAPE). Across the four datasets, joint-training produces a latent space organized by fitness and sequence information.

With reference to FIG. 4B, quantification of ruggedness is performed using a metric which measures neighborhood-level variation of a property in latent space. With reference to FIG. 4C - FIG. 4E, The ruggedness of representations was compared with respect to differences in fitness, λ_{f}, and differences in sequences, λₛ.

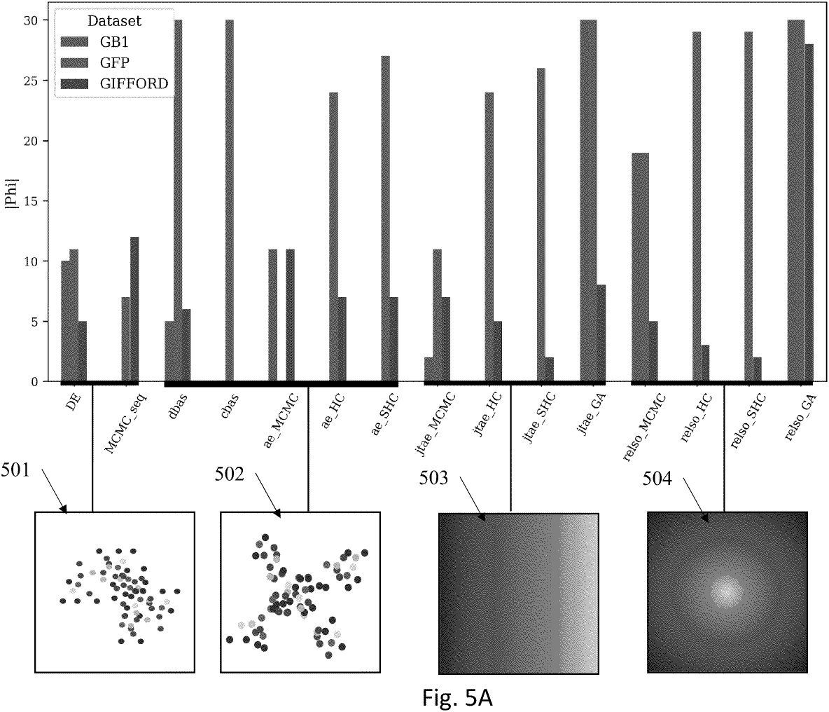

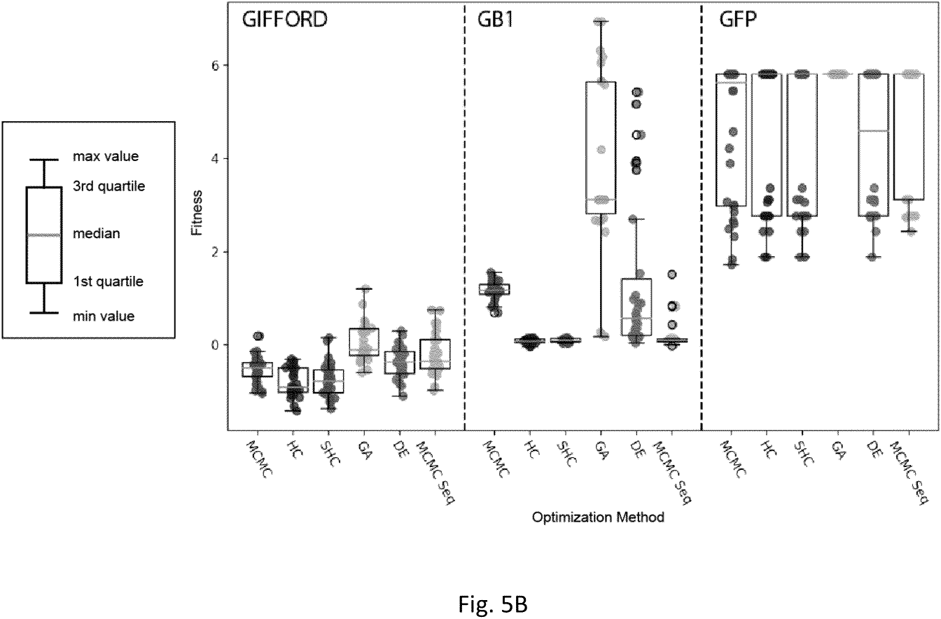

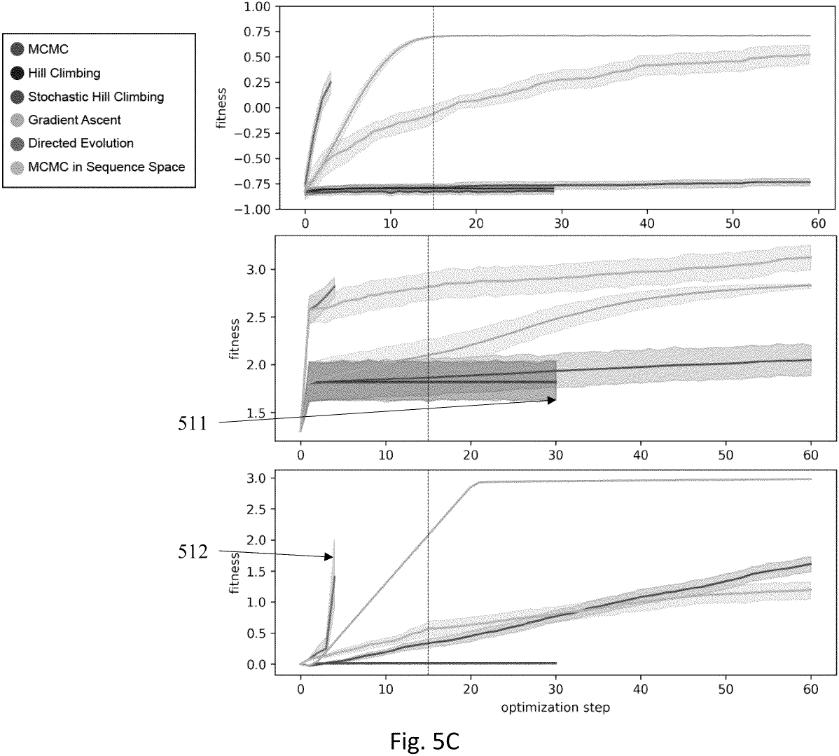

As improved sequences may possess hidden defects that present themselves in downstream analysis (e.g. immunogenicity of antibodies), it is often desired to produce several promising candidates at the end of the improvement stage. This scenario is replicated by collecting high-fitness sequences in a set ϕ whereby inclusion is restricted to sequences which are predicted to have a fitness value above some threshold. The considered improvement methods are evaluated by the cardinality of each method’s ϕ (see FIG. 5A) and the ending fitness values (see FIG. 5B). Additional results on the composition of sequences found in each method’s ϕ are included in Table 2 below, which includes a set of metrics which describe the quality and quantity of improved sequences generated. Each improvement approach began with a set of 30 seed sequences drawn from the bottom 25th percentile of each dataset’s held-out test split. Next, each improvement method was allowed an in-silico evaluation budget of 60 evaluations which corresponds to the number of sequences the method may observe and label. For methods like directed evolution which operate in a batchwise manner, the number of iterations was adjusted (see FIG. 5C) to keep the total observed sequences constant across methods.

TABLE 2

| Search Space | Algorithm | Max Fit. | Mean Fit. | Std Fit | \|ϕ\| | Novelty | Diversity | Ensgrad - Max | Ensgrad Mean |
|---|---|---|---|---|---|---|---|---|---|
| s | DE | 0.30 | -0.37 | 0.33 | 11 | 1.00 | 0.08 | -0.16 | -0.52 |
| | MCMC Seq | 0.75 | -0.18 | 0.45 | 13 | 1.00 | 0.12 | 0.12 | -0.53 |
| L-AE | MCMC | 0.00 | -0.39 | 0.23 | 11 | 1.00 | 0.08 | 0.23 | -0.06 |
| | HC | 1.35 | -0.62 | 0.51 | 7 | 0.14 | 0.03 | 0.25 | -0.64 |
| | SHC | 0.14 | -0.65 | 0.39 | 7 | 0.43 | 0.03 | -0.22 | -0.64 |
| L- JTAE | MCMC | 0.07 | -0.48 | 0.27 | 8 | 1.00 | 0.04 | -0.41 | -0.28 |
| | HC | 0.24 | -0.61 | 0.35 | 5 | 1.00 | 0.01 | -0.46 | -0.68 |
| | SHC | -0.13 | -0.70 | 0.28 | 1 | 0.00 | 0.00 | -0.02 | -0.70 |
| L - ReLSO | MCMC | 0.19 | -0.53 | 0.27 | 3 | 1.00 | 0.01 | 0.14 | -0.39 |
| | HC | -0.30 | -0.81 | 0.30 | 0 | NA | NA | -0.43 | -0.70 |
| | SHC | 0.15 | -0.76 | 0.36 | 2 | 0.00 | 0.00 | -0.35 | -0.69 |
| L-VAE | DBas | 0.00 | -0.47 | 0.25 | 6 | 1.00 | 0.01 | -0.27 | -0.58 |
| | CBas | -0.30 | -0.62 | 0.24 | 0 | NA | NA | -0.33 | -0.62 |
| L - JTAE | GA | -0.55 | -0.56 | 0.06 | 0 | 0.00 | 0.00 | -0.02 | -0.32 |
| L - ReLSO | GA | 1.20 | 0.05 | 0.41 | 23 | 1.00 | 0.24 | 0.33 | -0.01 |

It was found that ReLSO was able to produce a larger set of high-fitness sequences across the datasets with fewer steps. This was observed in FIG. 5C, where gradient ascent (GA) was able to efficiently reach to regions of high-fitness latent space and consequently generate high-fitness sequences with a fraction of the evaluation budget expended. With the narrow fitness landscape of GFP, it was observed that most methods are able to reach high-fluorescence sequences; however in GIFFORD and GB1, the performance gap between gradient ascent and other methods was more pronounced. Furthermore, in the case of GFP it was observed that all candidates of gradient ascent reside within the high-fluorescence mode of the dataset.

With reference to FIG. 5A, for each method, a batch of low-fitness sequences, sampled from the held-out set, were chosen as starting points. Ending improved sequences that were predicted to be high-fitness, determined by a threshold, are included in a set ϕ. The algorithms operate within a search space that is visualized below the plots, specifically sequence (501), AE (502), JTAE (503), and ReLSO (504). With reference to FIG. 5B, ending fitness values of all 30 seeds were plotted, split by dataset. With reference to FIG. 5C, the trajectories of fitness values over improvement step are shown, displaying a wide spread of efficiency. As some methods label multiple sequences during a single improvement step (e.g. hill climbing, with endpoint denoted as 511, and directed evolution, with endpoint denoted as 512) some lines do not reach across the entire x-axis. To highlight gradient ascent’s quick convergence to high-fitness, a vertical line is shown at the 15th step in each plot.

### Engineered Proteins

Implementations of the systems and methods discussed herein further provide or identify compositions comprising engineered proteins comprising one or more mutations that modify a desired trait or property of a sequence, for example a sequence encoding a protein, as compared to a trait or property of the native or parental protein. In one embodiment, the modified proteins generated or identified by implementations of the systems and methods discussed herein comprise one or more mutations at one or more amino acid residue predicted by the predictive pipeline of implementations of the systems and methods discussed herein to confer a desired trait or property or increase a fitness function of the protein.

The engineered proteins generated or identified by implementations of the systems and methods discussed herein may be made using chemical methods. For example, engineered proteins can be synthesized by solid phase techniques (Roberge J Y et al (1995) Science 269: 202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The engineered proteins may alternatively be made by translation of an encoding nucleic acid sequence, by recombinant means or by cleavage from a longer protein sequence. The composition of an engineered protein may be confirmed by amino acid analysis or sequencing.

The variants of the engineered proteins generated or identified by implementations of the systems and methods discussed herein may be (i) one in which one or more of the amino acid residues are substituted with a conserved or non-conserved amino acid residue and such substituted amino acid residue may or may not be one encoded by the genetic code, (ii) one in which there are one or more modified amino acid residues, e.g., residues that are modified by the attachment of substituent groups, (iii) fragments of the engineered proteins and/or (iv) one in which the engineered protein is fused with another protein or polypeptide. The fragments include polypeptides generated via proteolytic cleavage (including multi-site proteolysis) of an original engineered protein sequence. Variants may be post-translationally, or chemically modified. Such variants are deemed to be within the scope of those skilled in the art from the teaching herein.

As known in the art the “similarity” between two polypeptides is determined by comparing the amino acid sequence and its conserved amino acid substitutes of one polypeptide to a sequence of a second polypeptide. Variants are defined to include polypeptide sequences different from the original sequence, different from the original sequence in less than 40% of residues per segment of interest, different from the original sequence in less than 25% of residues per segment of interest, different by less than 10% of residues per segment of interest, or different from the original protein sequence in just a few residues per segment of interest and at the same time sufficiently homologous to the original sequence to preserve the functionality of the original sequence and/or the ability to bind to ubiquitin or to a ubiquitylated protein. Implementations of the systems and methods discussed herein may be used to generate or identify amino acid sequences that are at least 60%, 65%, 70%, 72%, 74%, 76%, 78%, 80%, 90%, 91%, 92%, 93%, 94%, 95%, 96%, 97%, 98%, or 99% similar or identical to the original amino acid sequence. The identity between two amino acid sequences is preferably determined by using the BLASTP algorithm [BLAST Manual, Altschul, S., et al., NCBI NLM NIH Bethesda, Md. 20894, Altschul, S., et al., J. Mol. Biol. 215: 403-410 (1990)].

The engineered proteins generated or identified by implementations of the systems and methods discussed herein can be post-translationally modified. For example, post-translational modifications that fall within the scope of implementations of the systems and methods discussed herein include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

An engineered protein generated or identified by implementations of the systems and methods discussed herein may be conjugated with other molecules, such as proteins, to prepare fusion proteins. This may be accomplished, for example, by the synthesis of N-terminal or C-terminal fusion proteins provided that the resulting fusion protein retains the functionality of the engineered protein.

### Engineered Protein Mimetics

In some embodiments, the subject compositions are peptidomimetics of the engineered proteins. Peptidomimetics are compounds based on, or derived from, peptides and proteins. The peptidomimetics generated or identified by implementations of the systems and methods discussed herein typically can be obtained by structural modification of a known engineered protein sequence using unnatural amino acids, conformational restraints, isosteric replacement, and the like. The subject peptidomimetics constitute the continuum of structural space between peptides and non-peptide synthetic structures; peptidomimetics may be useful, therefore, in delineating pharmacophores and in helping to translate peptides into non-peptide compounds with the activity of the parent engineered protein.

In addition to a variety of side chain replacements which can be carried out to generate the engineered protein peptidomimetics, implementations of the systems and methods discussed herein specifically contemplate the use of conformationally restrained mimics of peptide secondary structure. Numerous surrogates have been developed for the amide bond of peptides. Frequently exploited surrogates for the amide bond include the following groups (i) trans-olefins, (ii) fluoroalkene, (iii) methyleneamino, (iv) phosphonamides, and (v) sulfonamides.

### Nucleic Acids

In one embodiment, implementations of the systems and methods discussed herein may be used to generate or identify an isolated nucleic acid comprising a nucleotide sequence encoding an engineered protein.

The nucleotide sequences encoding an engineered protein can alternatively comprise sequence variations with respect to the original nucleotide sequences, for example, substitutions, insertions and/or deletions of one or more nucleotides, with the condition that the resulting polynucleotide encodes a polypeptide according to implementations of the systems and methods discussed herein. Accordingly, implementations of the systems and methods discussed herein may be used to generate or identify nucleotide sequences that are substantially identical to the nucleotide sequences recited herein and encodes a engineered protein.

In the sense used in this description, a nucleotide sequence is “substantially identical” to any of the nucleotide sequences describe herein when its nucleotide sequence has a degree of identity with respect to the nucleotide sequence of at least 60%, of at least 70%, at least 85%, at least 95%, at least 96%, at least 97%, at least 98% or at least 99%. A nucleotide sequence that is substantially homologous to a nucleotide sequence encoding an engineered protein can typically be isolated from a producer organism of the polypeptide generated or identified by implementations of the systems and methods discussed herein based on the information contained in the nucleotide sequence by means of introducing conservative or non-conservative substitutions, for example. Other examples of possible modifications include the insertion of one or more nucleotides in the sequence, the addition of one or more nucleotides in any of the ends of the sequence, or the deletion of one or more nucleotides in any end or inside the sequence. The identity between two nucleotide sequences is preferably determined by using the BLASTN algorithm [BLAST Manual, Altschul, S., et al., NCBI NLM NIH Bethesda, Md. 20894, Altschul, S., et al., J. Mol. Biol. 215: 403-410 (1990)].

In another aspect, implementations of the systems and methods discussed herein may be used to generate or identify a construct, comprising a nucleotide sequence encoding an engineered protein, or derivative thereof. In a particular embodiment, the construct is operatively bound to transcription, and optionally translation, control elements. The construct can incorporate an operatively bound regulatory sequence of the expression of the nucleotide sequence generated or identified by implementations of the systems and methods discussed herein, thus forming an expression cassette.

An engineered protein or chimeric engineered protein may be prepared using recombinant DNA methods. Accordingly, nucleic acid molecules which encode an engineered protein or chimeric engineered protein may be incorporated into an appropriate expression vector which ensures good expression of the engineered protein or chimeric engineered protein.

Therefore, in another aspect, implementations of the systems and methods discussed herein may be used to generate or identify a vector, comprising the nucleotide sequence or the construct generated or identified by implementations of the systems and methods discussed herein. The choice of the vector will depend on the host cell in which it is to be subsequently introduced. In a particular embodiment, the vector generated or identified by implementations of the systems and methods discussed herein is an expression vector. Suitable host cells include a wide variety of prokaryotic and eukaryotic host cells. In specific embodiments, the expression vector is selected from the group consisting of a viral vector, a bacterial vector and a mammalian cell vector. Prokaryote- and/or eukaryote-vector based systems can be employed for use with implementations of the systems and methods discussed herein to produce polynucleotides, or their cognate polypeptides. Many such systems are commercially and widely available.

Further, the expression vector may be provided to a cell in the form of a viral vector. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

Vectors suitable for the insertion of the polynucleotides are vectors derived from expression vectors in prokaryotes such as pUC18, pUC19, Bluescript and the derivatives thereof, mp18, mp19, pBR322, pMB9, ColE1, pCR1, RP4, phages and “shuttle” vectors such as pSA3 and pAT28, expression vectors in yeasts such as vectors of the type of 2 micron plasmids, integration plasmids, YEP vectors, centromere plasmids and the like, expression vectors in insect cells such as vectors of the pAC series and of the pVL, expression vectors in plants such as pIBI, pEarleyGate, pAVA, pCAMBIA, pGSA, pGWB, pMDC, pMY, pORE series and the like, and expression vectors in eukaryotic cells based on viral vectors (adenoviruses, viruses associated to adenoviruses such as retroviruses and, particularly, lentiviruses) as well as non-viral vectors such as pSilencer 4.1-CMV (Ambion), pcDNA3, pcDNA3.1/hyg, pHMCV/Zeo, pCR3.1, pEFI/His, pIND/GS, pRc/HCMV2, pSV40/Zeo2, pTRACER-HCMV, pUB6/V5-His, pVAX1, pZeoSV2, pCI, pSVL and PKSV-10, pBPV-1, pML2d and pTDT1.

By way of illustration, the vector in which the nucleic acid sequence is introduced can be a plasmid which is or is not integrated in the genome of a host cell when it is introduced in the cell. Illustrative, non-limiting examples of vectors in which the nucleotide sequence or the gene construct generated or identified by implementations of the systems and methods discussed herein can be inserted include a tet-on inducible vector for expression in eukaryote cells.

In a particular embodiment, the vector is a vector useful for transforming animal cells.

The recombinant expression vectors may also contain nucleic acid molecules which encode a portion which provides increased expression of the engineered protein or chimeric engineered protein; increased solubility of the engineered protein or chimeric engineered protein; and/or aid in the purification of the engineered protein or chimeric engineered protein by acting as a ligand in affinity purification. For example, a proteolytic cleavage site may be inserted in the engineered protein to allow separation of the engineered protein or chimeric engineered protein from the fusion portion after purification of the fusion protein. Examples of fusion expression vectors include pGEX (Amrad Corp., Melbourne, Australia), pMAL (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

The invention provides a method which maps biomolecular sequences to an embedding space within a deep, regularized autoencoder where improvements can more efficiently take place. The model is also generative, allowing for the generation of novel sequences from latent space. This effectively transforms the original discrete improvement problem to a continuous one. The deep encoder is based on a transformer architecture and therefore also confers both a powerful representation learning ability as well as a path towards interpretability via attention map analysis.

A core novelty the invention is that the improvement takes place within the encoding space of a deep encoder, rather than directly in sequence space. This allows greater control over the qualities of the space where improvements takes place. Novel regularizations are introduced to this model which make this latent space improvement approach possible. Lastly the use of a transformer within this jointly-trained autoencoder framework is also novel.

The model can prioritize screening candidates as well as propose new candidates. Furthermore, the gradient-based approach to searching for candidates allows for computational efficiency gains over alternative approaches (Bayesian optimization, MCMC).

### Example 1: Deep Learning-Based Design and Improvement Methods for Next Generation Therapeutic Antibodies

Biomolecules can display a wide range of functions and properties, making them a highly versatile class of therapeutic. This valuable characteristic is a function of sequence and structure. Biomolecules find use in applications including gene therapy, antibodies, and as small molecule therapeutics (e.g., as kinase inhibitors). However, there is a high research and development cost for bringing biomolecules to the market (e.g., approx. 700 million/antibody drug.)

The therapeutic antibody marketplace is rapidly increasing with an increasing number of antibody therapies available.

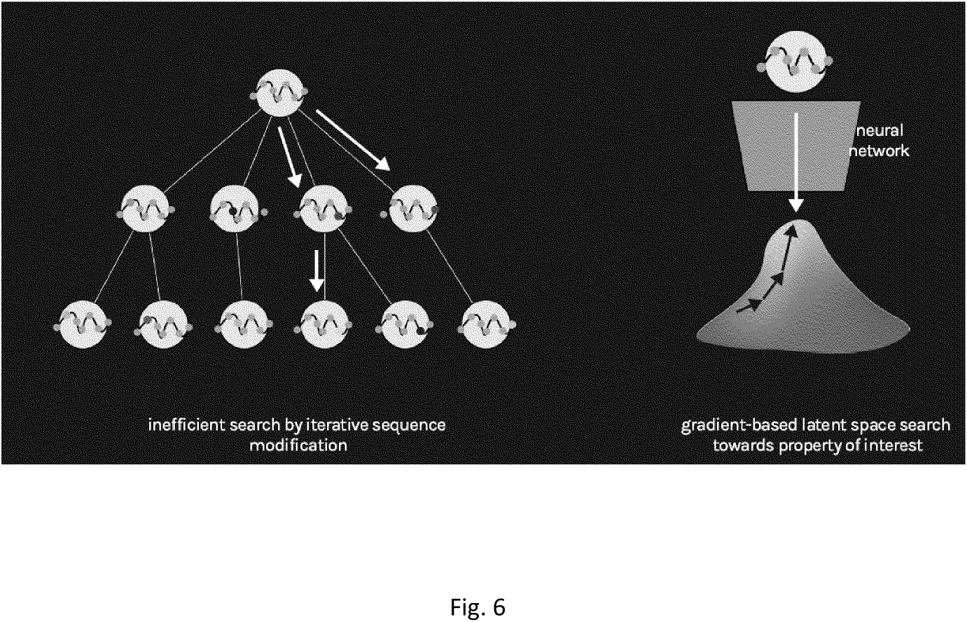

The invention provides a platform that can produce the next generation of antibody therapeutics with unprecedented efficiency (FIG. 1A and FIG. 1B). In contrast to methods that use iterative sequence modification, an inefficient search mechanism, the method of the invention employs a gradient-based latent space search towards a property of interest (FIG. 6).

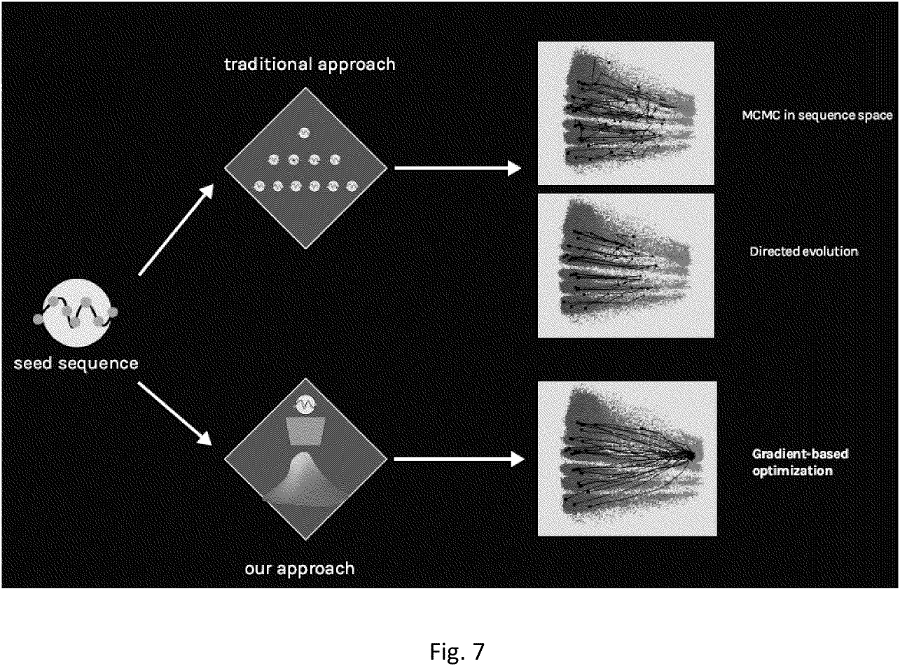

The method of the invention was used to improve complementarity determining regions of human Immunoglobulin G. The data set was sourced from Li et al., (Li et al., Bioinformatics 36.7 (2020): 2126-2133.) The method of the invention provided a more efficient method for navigating though sequence space by way of latent space (FIG. 7).

### Example 2 - Improvement of Anti-Ranibizumab Antibodies

A key high-throughput phage panning dataset used for antibody improvement and generation was obtained from Liu, G. et al., Bioinformatics (2020). This dataset provided approximately 60,000 samples for CDR-H3 sequence improvement against the ranibizumab antibody. Success in this task has implications in the context of modifying antibody binding to respective targets, thus further improving antibody-antigen binding, or generative antibody design with reduced anti-drug antibody affinity. Together, this dataset motivated the task of localized manipulation of the CDR-H3 region of anti-ranibizumab sequences within the latent space of the disclosed model.

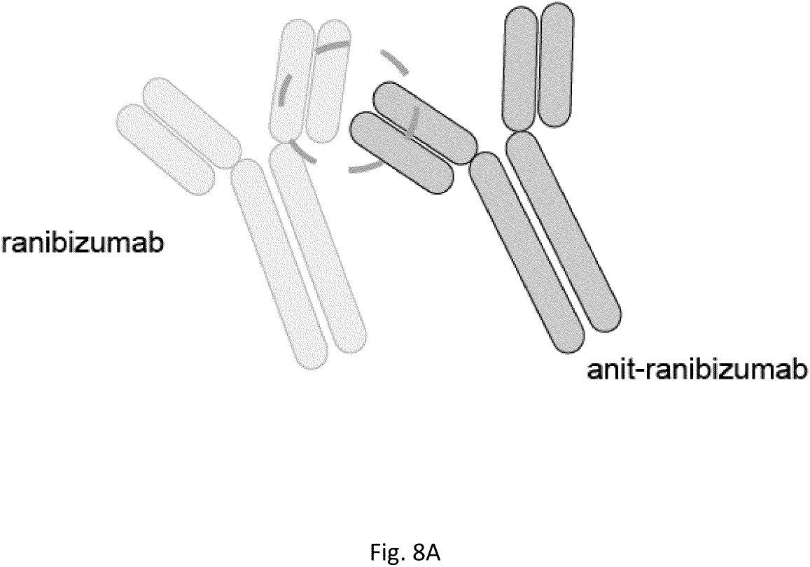

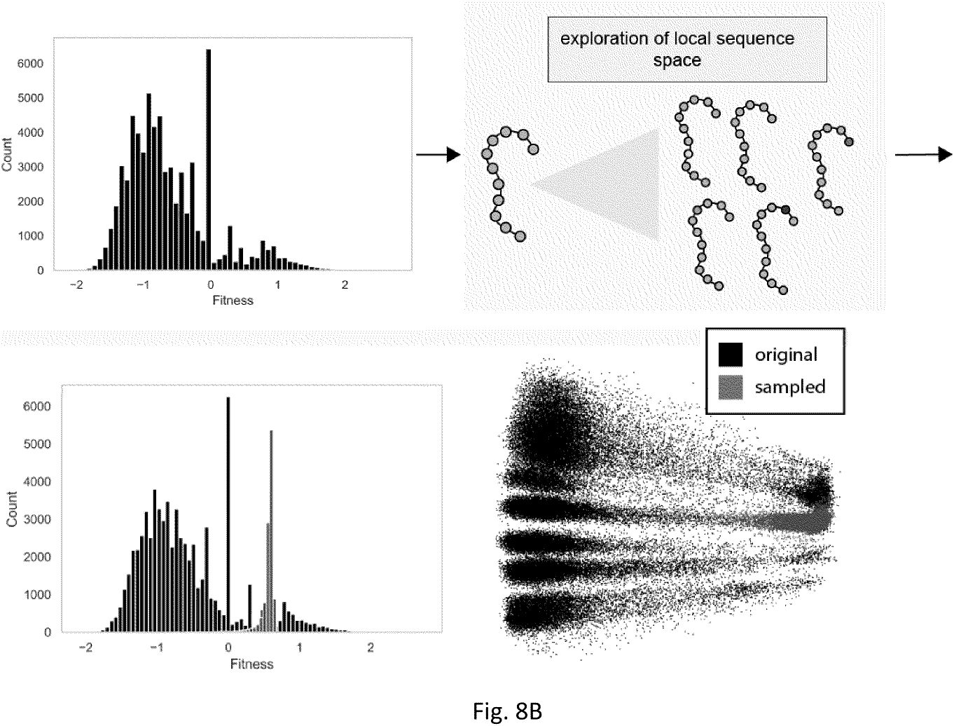

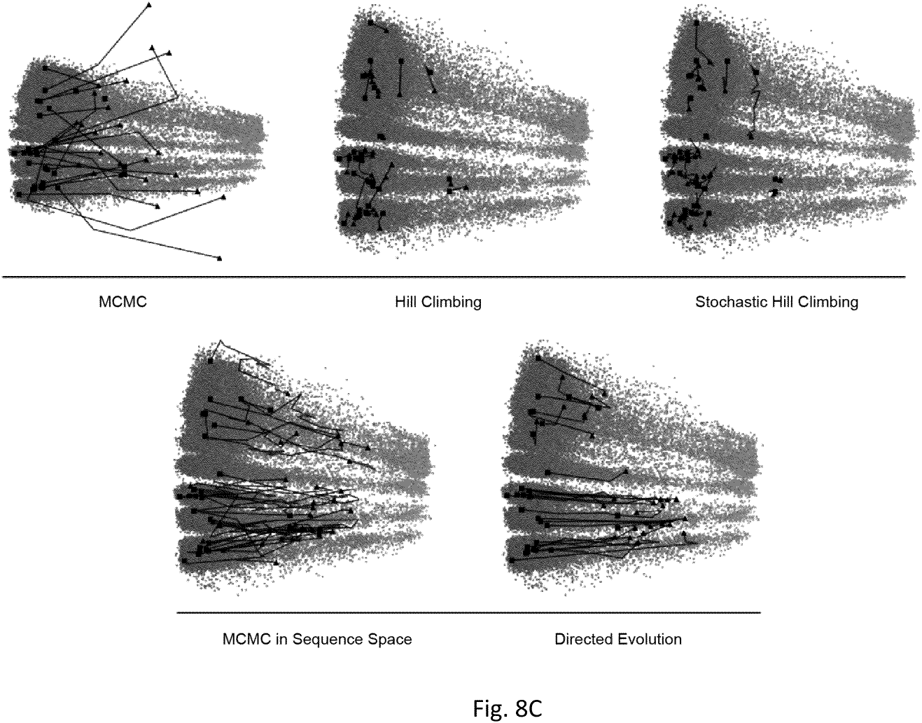

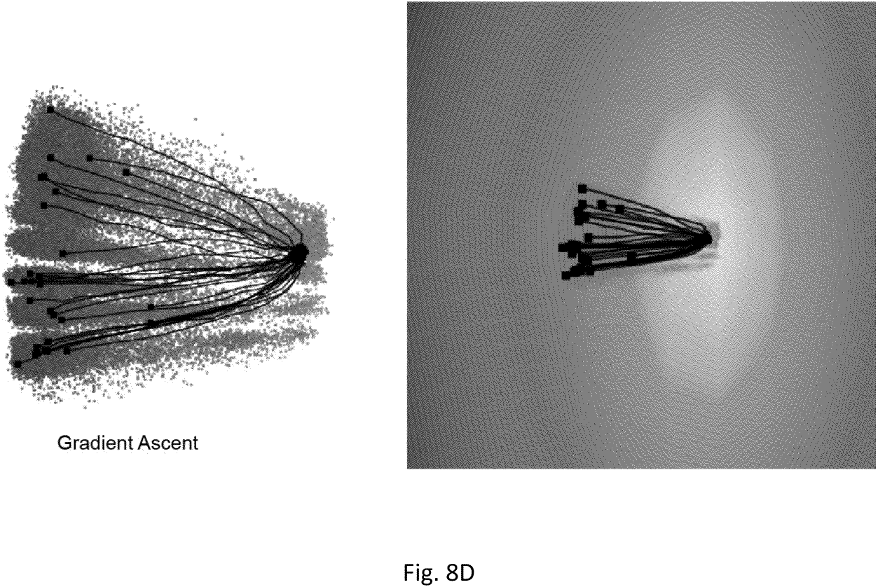

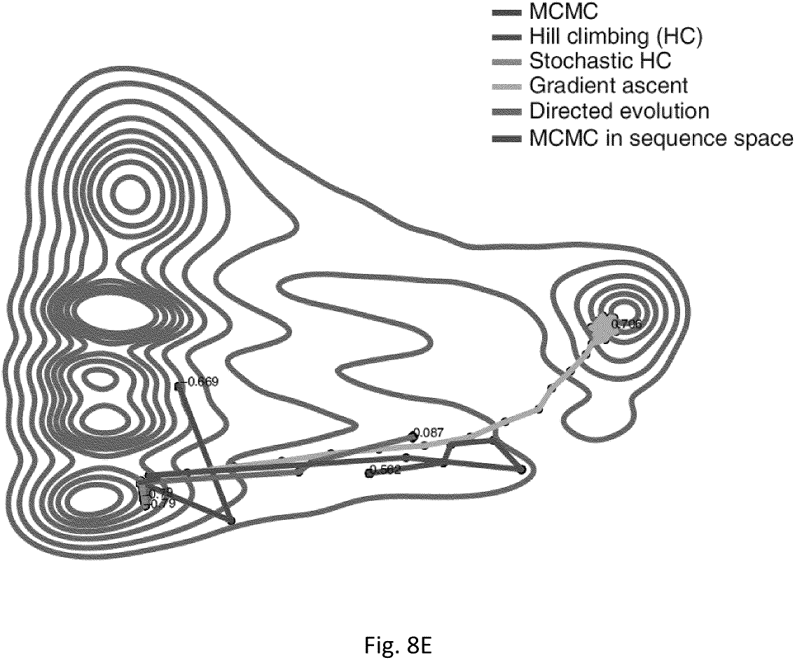

To subsequently explore the ability of the model to generalize to similar but nonidentical sequences in the latent space, 11,061 sequences were generated using the top sequences within the Gifford dataset (enrichment binding affinity ≈1.5) (see FIG. 8B). This filtered to 43 high fitness ‘seed’ antibody sequences. Given the variability in physicochemical properties with respect to the potential of a single amino acid, variants were generated from each of these 43 sequences at one mutational amino acid distance away from these 43 starting sequences. This allowed exploration of the local neighborhood of the high fitness latent space while also localizing to a particular window of the CDR3 domain.

Upon interpolation of the high fitness sequences, each of 11,061 sequences was passed into the trained model for both binding affinity and embedding predictions. These embeddings and predicted fitness values were then examined (see FIG. 8B). Interestingly, the generated sequences localized to a single high fitness region of the latent space. Additionally, the generated sequence abundance appeared to be associated with a relatively high level of fitness as shown in FIG. 8B, in line with the learned organization by fitness in latent space from the initial training data. These generative results suggest the potential implication of low mutational distances from regions of interest within the latent space of encoded sequences. Furthermore, latent space improvement steps followed by a more dense, local generative sampling of high fitness variants may suggest an interesting avenue for sequence space exploration and bio-therapeutic improvements.

Next, the improvement trajectories taken by each improvement method were examined across seed sequences. As previously described, each improvement begins with a low-fitness seed sequence and all methods have an equal in-silico labeling budget (i.e. calls to a fitness oracle). It was observed that gradient-free methods, operating in either latent space or sequence space, all exhibit an inherent level of inefficiency in their search for higher fitness (see FIG. 8C). For Markov Chain Monte Carlo (MCMC) in latent space, this is compounded by the departure of improvement trajectories into sparsely populated regions of latent space. In contrast, the ability to leverage fitness gradients enables a more robust approach to sequence design whereby each improvement trajectory is able to reach high-fitness regions of latent space and be decoded to improved sequences (see FIG. 8D). To highlight the difference in improvement efficiency between methods, the improvement trajectories were plotted across several methods for a single seed in FIG. 8E.

With reference to FIG. 8A - FIG. 8E, various diagrams related to improving anti-ranibizumab antibodies are shown. With reference to FIG. 8A, this task is focused on improving the highly-variable CDR3 region (circled) of the anti-ranibizumab antibody. With reference to FIG. 8B, the tight coupling between Euclidean distance in latent space, sequence distance, and fitness difference is examined. A set of sequences were generated from the local mutation space around a small set of high fitness seed sequences present in the GIFFORD training set. These newly generated sequences were then encoded into the latent space of ReLSO and fitness values were predicted by the model. FIG. 8C depicts a visualization of improvement paths taken by gradient-free methods compared with those in this study. Each trajectory starts at a low-fitness sequence sampled from the held-out test set. FIG. 8D depicts improvement paths taken by gradient ascent, which converge to a high-fitness region of latent space. This is a result of the underlying regularized fitness function learned by ReLSO. FIG. 8E shows that, for the improvement of a single seed, paths taken by gradient-free methods show a less-efficient progression to the data-driven global fitness maxima.

### Example 3 - Green Fluorescent Protein (GFP) Landscape

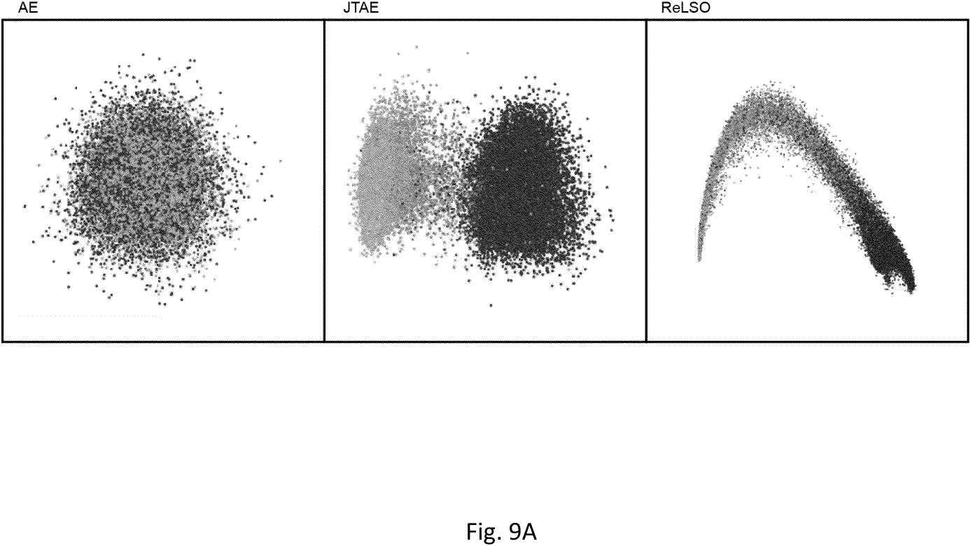

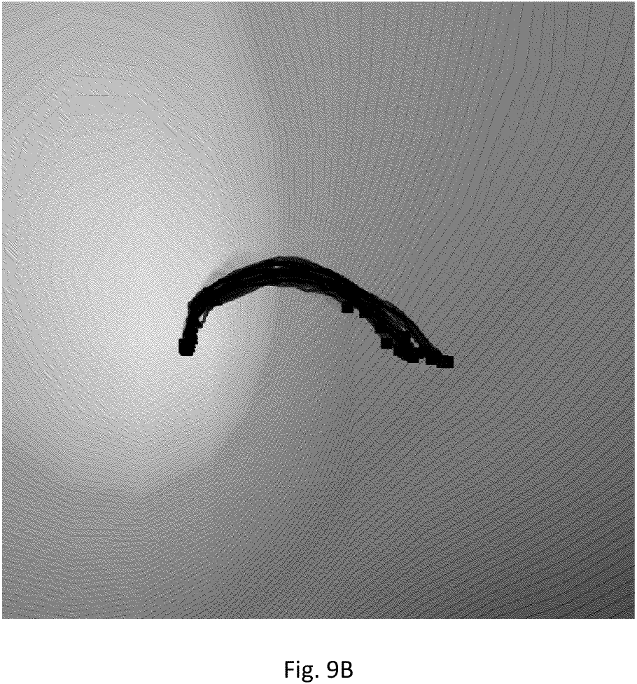

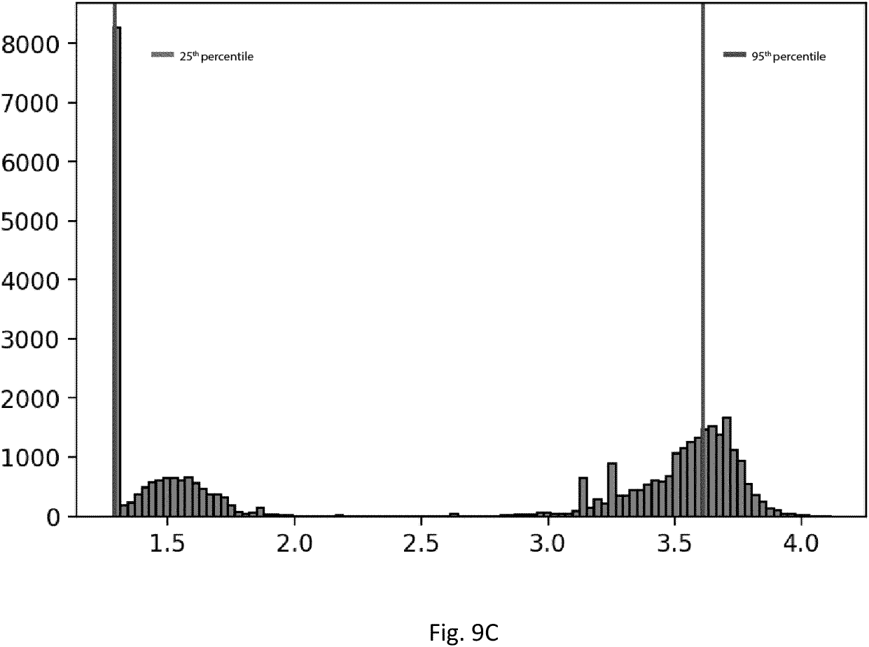

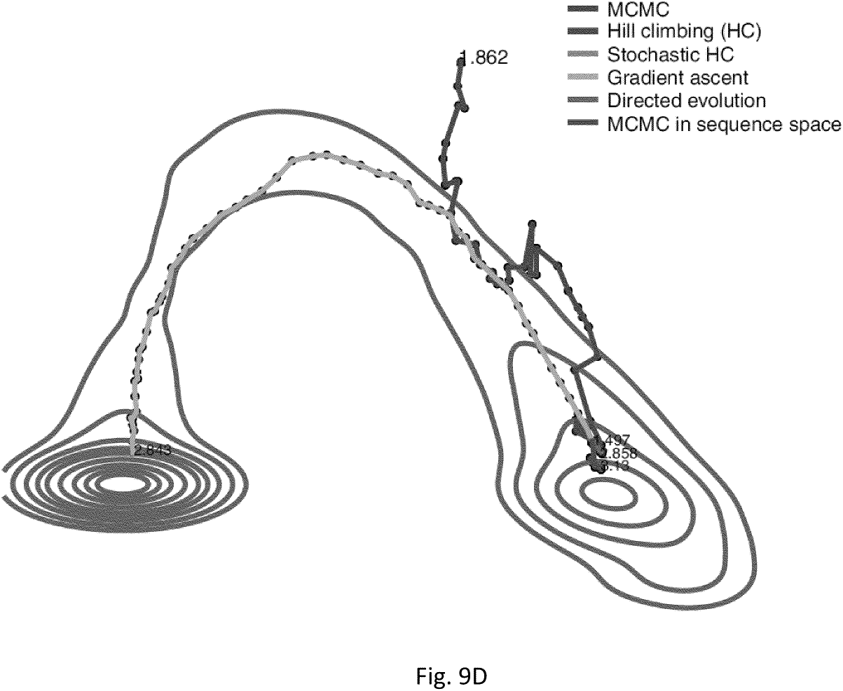

With the latent space organized by fitness, as demonstrated by both visualization of the latent coordinates and the learned fitness function, in-silico sequence improvements were conducted with the same setup used on the GIFFORD dataset. First seed sequences were sampled from the held-out test set and sequences were selected from the bottom quartile of observed fitness (log fluorescence ≤ 1.3) as visualized by the red vertical line in FIG. 9C. The threshold for high-fitness and inclusion in ϕ was set at the 95th percentile of log fluorescence (3.76) and is visualized with the green vertical line in FIG. 9C. Evaluation of improvement methods was then carried out and the results are presented in FIG. 5A - FIG. 5C and Table 3 below. A quick convergence was observed to high-fluorescence sequences by several methods, likely owing to the narrowness of the underlying fitness landscape. However, knowledge of the fitness landscape still provides a reliable route for improvement as not all seed sequences were able to be successfully improved by methods other than gradient ascent (see FIG. 9D). For sequences predicted to be high-fluorescence by ReLSO, a stabilizing change made to the sequence was observed (avg ddG = -1.3 kcal/mol) as determined by Dynamut2 (Rodrigues, C. H., et al., Protein Sci. (2021)).

TABLE 3

| | GIFFORD | | | GB1 | | | GFP | | |
|---|---|---|---|---|---|---|---|---|---|
| | λ_{f} | λₛ | λ^{^} | λ_{f} | λₛ | λ^{^} | λ_{f} | λₛ | λ^{^} |
| Sequence | 1.47 | 1.49 | 1.48 | 0.99 | 0.09 | 0.54 | 8.42 | 0.07 | 4.25 |
| AE | 1.70 | 1.96 | 1.83 | 1.00 | 0.09 | 0.54 | 7.52 | 0.10 | 3.81 |
| TAPE | 1.91 | 2.08 | 2.00 | 0.86 | 1.98 | 1.42 | 5.09 | 0.10 | 3.09 |
| JT-AE | 1.38 | 2.03 | 1.70 | 0.05 | 0.11 | 0.08 | 0.88 | 0.12 | 0.50 |
| TAPE + finetune | 1.53 | 2.96 | 2.24 | 1.64 | 0.33 | 0.99 | 11.17 | 0.16 | 5.67 |
| ReLSO (interp) | 1.36 | 2.03 | 1.70 | 0.04 | 0.11 | 0.08 | 7.20 | 0.11 | 3.65 |
| ReLSO (neg) | 1.39 | 2.06 | 1.72 | 0.05 | 0.11 | 0.08 | 1.80 | 0.15 | 0.97 |
| ReLSO α = 0.1 | 1.83 | 1.96 | 1.89 | 0.40 | 0.10 | 0.25 | 1.15 | 0.11 | 0.63 |
| ReLSO α = 0.5 | 1.33 | 2.00 | 1.67 | 0.07 | 0.11 | 0.09 | 0.96 | 0.12 | 0.54 |
| ReLSO | 1.36 | 2.05 | 1.70 | 0.05 | 0.11 | 0.08 | 3.68 | 0.16 | 1.92 |

In an effort to empirically describe epistasis in the fitness landscape of GFP, Sarkisyan, K. S. et al., Nature (2016) performed random mutagenesis of the Aequorea victoria GFP proteinto produce 56,086 variants. The fluorescence of each variant was quantified, with multiple steps taken to ensure accurate estimates. The dataset produced from this study, which includes a mixture of variants with an average of 3.7 mutations per sequence, exhibited a narrow fitness landscape and a bimodal distribution of fluorescence values (see FIG. 9C). The sequence-fitness relationship in the dataset presented an opportunity to evaluate the disclosed model on a protein known to have epistastic relationships between residues and a challenging improvement landscape. The ruggedness of the fitness landscape was observed in the sequence representation and latent representation generated by the AE model by principal component analysis (see FIG. 4A, FIG. 4B). However in the latent representations of JT-AE and ReLSO the bimodal distribution of fitness in GFP fitness landscape is recapitulated (see FIG. 9A). Investigation of the fitness function learned by the fitness prediction network of ReLSO, h_{θ}, revealed that the model had learned a smoothly changing function from low to high fluorescence and a global maximum in the high-fluorescence mode (see FIG. 9B).

With reference to FIG. 9A - FIG. 9D, various visualizations related to improvement of fluorescent brightness of a GFP are shown. FIG. 9A shows a visualization of the latent embeddings produced by AE, JT-AE, and ReLSO models on the GFP dataset, colored by log fluorescence. FIG. 9B shows the learned fitness function extracted from the auxiliary fitness prediction network of ReLSO. FIG. 9C shows a bimodal distribution of log fluorescence values with low and high fluorescence modes. FIG. 9D shows improvement paths of a single, low-fluorescence seed sequence using various gradient-free and gradient-based methods.

### Interpretability

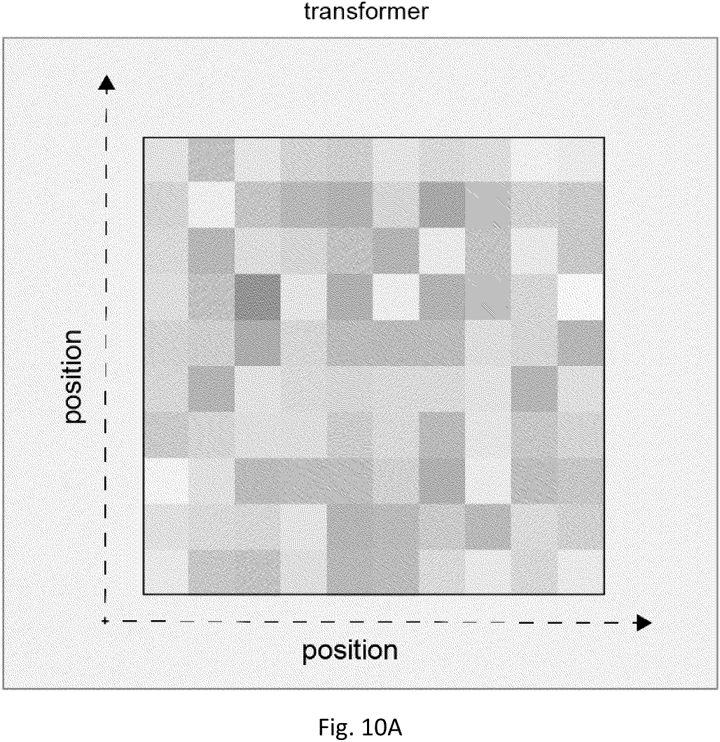

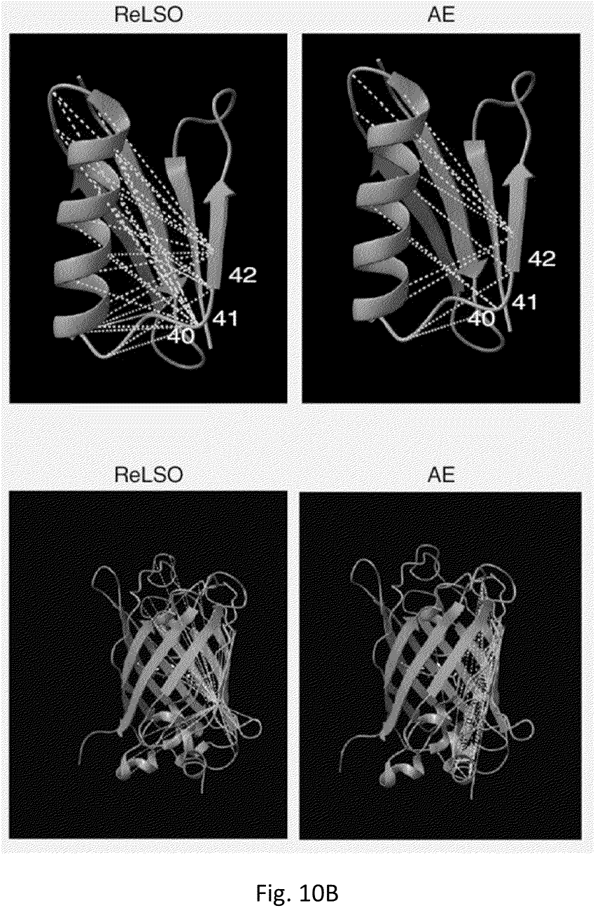

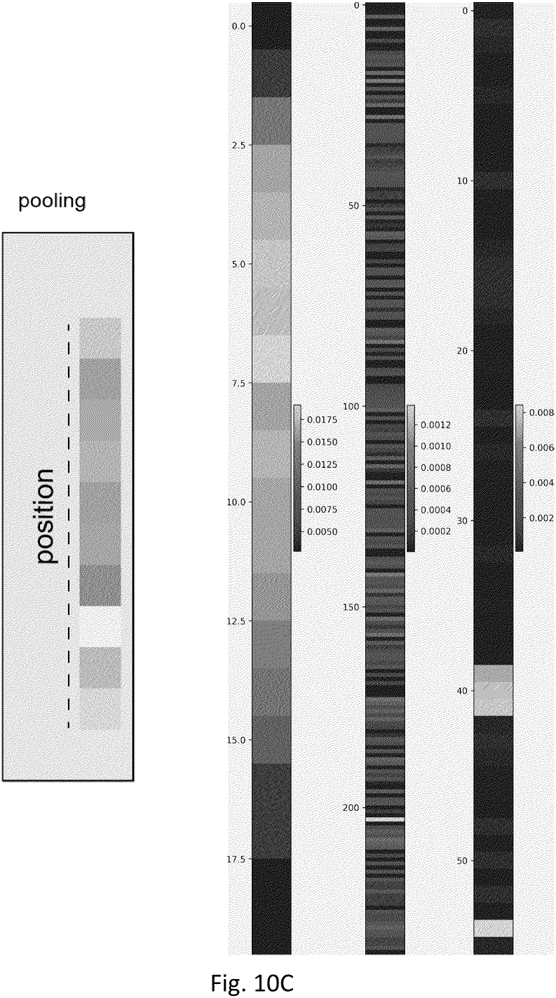

Encouraged by the success of other works, the attention weightings of the trained ReLSO model were examined for possible localized sequence-fitness attribution. It was hypothesized that given the joint-training approach and the observed organization by fitness in the latent embeddings (FIG. 4B), the learned attention information from the ReLSO model may also aid in elucidating fitness-sequence relationships. As the model displays a high level of performance on fitness prediction and sequence reconstruction (see Table 4A and Table 4B below) it was posited that the model captured salient and predictive aspects of protein sequences in its trained parameters. To examine this, attention information was extracted from both the attention maps of the transformer layers (see FIG. 10A) and the attention-based pooling operation. The former provides pairwise attention information while the latter provides positional attention information. To extract attention information, sequences were randomly sampled from a dataset and passed through the encoder and bottleneck modules of ReLSO. In each forward-pass, the attention maps of transformer model and the pooling weights of the bottleneck module were kept and aggregated. Averaging was then performed to reduce the level of noise. Additionally a thresholding step was performed on the averaged attention maps to increase sparsity such that only the most salient relationships remained.

TABLE 4A

| | Gifford | | | | GB1 | | | |
|---|---|---|---|---|---|---|---|---|
| | Task 1 | | Task 2 | | Task 1 | | Task 2 | |
| | Perplexity | Accuracy | MSE | Spearman ρ | Perplexity | Accuracy | MSE | Spearman ρ |
| AE | 1.03 | 0.90 | 0.88 | -0.15 | 1.00 | 1.00 | 0.17 | 0.00 |
| JT-AE | 1.21 | 0.82 | 0.22 | 0.47 | 1.00 | 1.00 | 0.01 | 0.43 |
| ReLSO (interp) | 1.21 | 0.82 | 0.22 | 0.48 | 1.00 | 1.00 | 0.01 | 0.43 |
| ReLSO (neg) | 1.24 | 0.81 | 0.29 | 0.47 | 1.00 | 1.00 | 0.02 | 0.42 |
| ReLSO α = 0.1 | 1.02 | 0.91 | 0.72 | 0.35 | 1.00 | 1.00 | 0.09 | 0.53 |
| ReLSO α = 0.5 | 1.07 | 0.88 | 0.34 | 0.50 | 1.00 | 1.00 | 0.02 | 0.45 |
| ReLSO | 1.17 | 0.84 | 0.29 | 0.48 | 1.00 | 1.00 | 0.01 | 0.44 |

TABLE 4B

GFP

| | Task 1 | | Task 2 | |
|---|---|---|---|---|
| | Perplexity | Accuracy | MSE | Spearman ρ |
| AE | 1.00 | 0.99 | 6.74 | 0.13 |
| JT-AE | 1.04 | 0.99 | 0.18 | 0.85 |
| ReLSO (interp) | 1.03 | 0.99 | 0.13 | 0.86 |
| ReLSO (neg) | 1.09 | 0.98 | 0.22 | 0.77 |
| ReLSO α = 0.1 | 1.03 | 0.99 | 0.18 | 0.84 |
| ReLSO α = 0.5 | 1.04 | 0.99 | 0.12 | 0.85 |
| ReLSO | 1.10 | 0.98 | 0.52 | 0.70 |

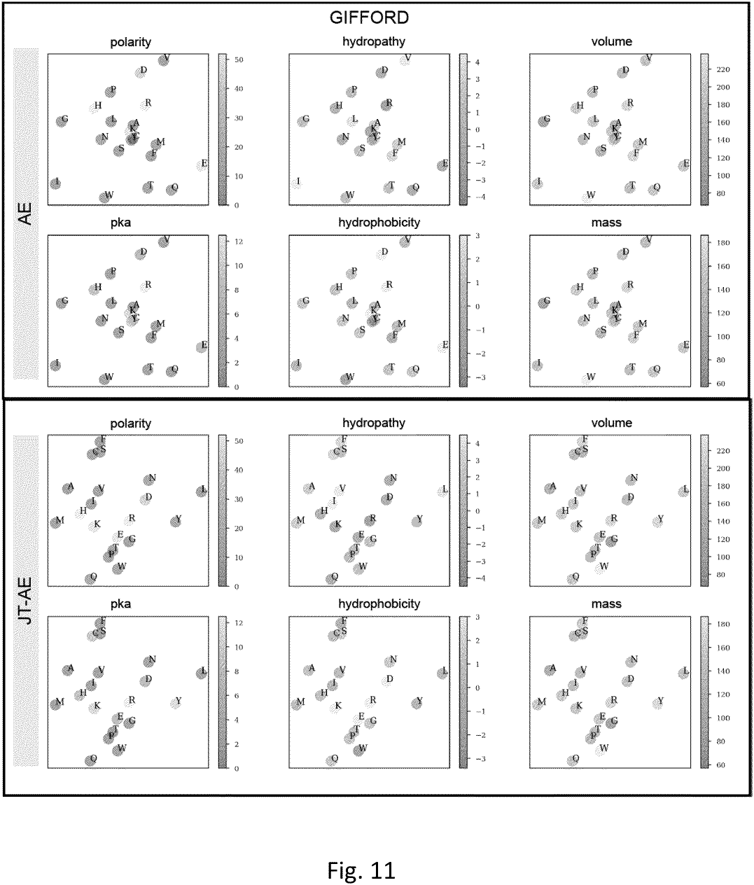

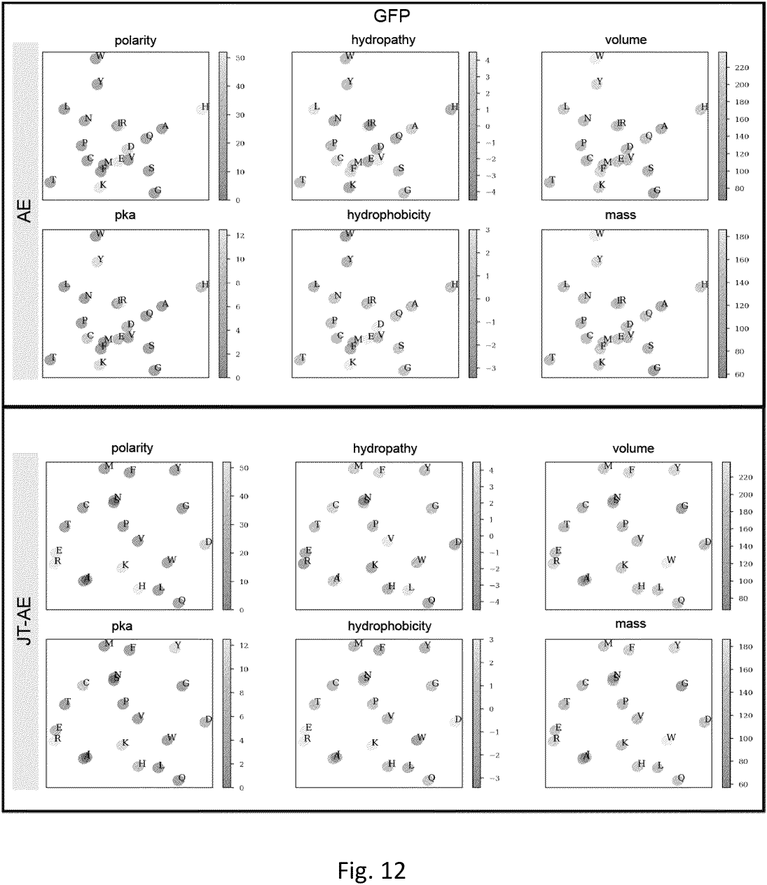

As previous approaches have focused on transformers trained in an unsupervised or self-supervised manner, the attention information was compared between AE and ReLSO (see FIG. 10B). Several differences were observed between the learned attention relationships of the ReLSO models and the AE models across the datasets. This divergence of attention relationships was viewed as a potential signal that the attention maps of transformers trained to predict fitness may also be probed to study sequence-fitness and structure-fitness relationships. Interestingly, the attention maps for GFP learned by ReLSO indicate the presence of potential epistatic interactions across a cylindrical pocket of the protein. Such long-range epistatic interactions have previously been identified in GFP by experts in the field. Automated discovery of such interactions via the ReLSO architecture presents an exciting avenue for follow-up investigation. Next, it was observed that the variable residues in the GB1 dataset (39-41, 54) are highly-weighted in the positional attention extracted from the ReLSO model, as shown in FIG. 10C. As these 4 positions are the only changing residues across sequences in the GB 1 dataset, their importance for the prediction tasks was confirmed with their attention maps as expected. In addition, the embeddings of the amino acids recapitulate their underlying biochemical properties in their organization, shown in FIG. 11 and FIG. 12. This result was shared in previous work as a signal that the model has learned biologically meaningful information in its parameters (Rives, A. et al., Proc. Natl Acad. Sci. (2021)).

### Discussion

The ability to find better representations is vital to extracting insights from noisy, high-dimensional data within the fields of protein biology. Defined by their biochemical interactions, evolutionary selection pressures, and function-stability tradeoffs, proteins are an increasingly important domain for the application of deep learning. More specifically, the field of biotherapeutic development has recently shown significant benefits from the application of both linear and non-linear models. Some of the very impactful models in this space have been largely supervised, but more recent work has proven the usefulness of leveraging unsupervised learning to pre-train predictive models to identify protein sequences with an enhanced property of interest.

The disclosed method took an alternative path combining these two types of learning objectives by instead taking a multi-task learning approach. Through simultaneously targeting protein sequence generation and fitness level prediction, a latent space rich in information about both sequence and fitness information was enforced. Importantly, this fitness information may encompass a variety of different properties such as binding affinity and fluorescence, which are smoothly embedded in the latent space of the trained model. Regularizations were then added that reflect principles of protein engineering, reshaping the latent space in the process. Leveraging these regularizations and the architecture of the model, it was shown that gradient ascent can deliver improvements in proteins when searching over the protein sequence space.

The departure of this approach from other methods demonstrates a novel and promising avenue for improving the ability to design and improve proteins. Furthermore, the reliance of this method solely on sequence information paired to a fitness value suggests that ReLSO-like architectures can be applied to other biomolecules such as DNA and RNA. In particular, one application to nucleic acids would be to improve gene editing tools such as CRISPR-Cas9 to reduce off-target effects. Specifically, in some embodiments, a method may tune binding affinity to increase selectivity towards a certain target or isoform, but against others to mitigate off-target toxicity. With the growing prominence of biological therapeutics, the disclosed methods have potential to deliver improvements in the development of improved therapeutics.

### Methods

ReLSO can be understood as being comprised of four main modules: the Encoder Module, the Bottleneck Module, the Sequence Prediction Module, and the Fitness Prediction Module. The encoder module takes as input protein sequences, encoded as an array of token indices, and outputs an array of token embeddings. The encoder module may in some embodiments be a transformer with 10 layers and 4 heads/layer. In other embodiments, the encoder module may have between 4 and 20 layers, between 6 and 16 layers, between 8 and 12 layers, at least 8 layers, at least 10 layers, or at most 10 layers. A token embedding size of 300 and a hidden size of 400 was used in this disclosure.

Next, amino acid-level embeddings are passed through a bottleneck module which in some embodiments is made up of two fully-connected networks. In one embodiment, the first network reduces the dimensionality of each token to the latent dimension, 30, whereas the second predicts a vector of weights summing to 1. In other embodiments, different latent dimensionality may be used, including but not limited to 2, 3, 4, 5, 10, 15, 20, 40, 50, 60, 70, 100, 200 dimensions, or any number of dimensions in the range of 10 to 50 or 2 to 200. The outputs of these two networks are then combined in a pooling step where a weighted sum is taken across the 30-dimensional embeddings to produce a single sequence-level representation, z _{E} R³⁰. This representation is of focus in the present disclosure and is referred to as a protein sequence’s latent representation.

To decode latent points to sequences, a deep convolutional network is used, comprised of four one-dimensional convolutional layers. In other embodiments, alternative numbers of convolutional layers may be used, for example 2, 3, 5, 6, 7, 8, 10, or more convolutional layers. ReLU activations and batchnorm layers may be used between convolutional layers with the exception of the final layer.

The final module is a fitness prediction network which predicts fitness from points in latent space. To encourage gradual changes to fitness in latent space, a 2-layer fully connected network regularized by a spectral norm penalty was used, introduced in Yoshida, Y. & Miyato, T., https://arxiv.org/abs/1705.10941 (2017)). As a result, the network is further encouraged to learn simpler organizations such as the pseudo-concave shape disclosed herein.

Each model was trained for 300,000 steps with a learning rate of 0.00002 on two 24 GB TITAN RTX GPUs, using a batch size of 64. For the negative sampling and interpolative sampling regularizations, samples of 32 artificial latent points were used in each forward pass bringing to total effective batch size to 128.

From a preliminary experimental screen of variants, a set of protein sequences X, where each sequence is a ordered set of amino acids x = (σ₁, σ₂, ..., ^{σ}_{N-1}, ^{σ}_{N}) composed from a finite alphabet of amino acids such that ^{σ}ᵢ C. V, i ∈ [N] and their corresponding fitness values y, y ∈ R is produced. The final dataset D is then comprised of pairs (x_{i,} _{Y}i) of sequences and their observed fitness. From this data, it is desirable to find sequences x* _{E} S that possess a high degree of fitness, as measured by some threshold {y* >_ _{Ythresh} _{I} y* = Φ (x *), x _{*}∈ ϕ}. It is also desirable that solutions in ϕ be diverse and novel.

The disclosed methods were formulated by first starting at the traditional perspective of sequence-based protein design. While directed evolution has yielded various successes in a range of domains over the years, it is susceptible to the underlying topology of the fitness landscape. This may lead to the accumulation of only locally optimal sequences at the completion of the improvement process. Recent work has sought to overcome the screening burden of directed evolution by performing in-silico evaluations of a candidate sequence’s fitness. This approach is comprised of training a model ϕ̂x to approximate the “ground-truth” fitness landscape ϕₓ by minimizing an objective function of the form L = | |y - yl 1, where y = ϕₓ(x) and y = ϕₓ(x). Once the model has converged, it is then used to evaluate sequence candidates x in either using either an iterative modification or sampling approach. In either case, the local sequence space around the original sequence is explored using minor changes to x, Δₓ. However, the difficult to predict relationship between Δₓ and changes in fitness Δ_{y} maintains the challenging nature of improvement within sequence space.

A more recent approach to sequence-based protein design is to train a deep learning model to learn a representation of protein sequences by pre-training the model on a large corpus of protein sequence data with an unsupervised task ∥g(f(x)) - x∥ where f_{θ} is an encoder and g_{θ} is a decoder. The end result of this pre-training is a trained encoder that has learned a function z = f_{θ}(x), where z is understood to contain abstract and useful information about protein sequence composition. For the unsupervised model, it can be further stated that learned latent code approximates the manifold on which the training data lies, where higher density is placed on realistic sequences.

Next, a prediction model h_{θ} is trained on the latent representation z to learn a fitness landscape y = Φ̅_{Z} = h_{θ}(z) that will be used to evaluate new candidates x̅. While this approach moves the improvement problem to a continuous setting, it suffers from an issue inherent in datasets from this domain. Often the datasets gathered from screening contain a small percentage of high-fitness sequences while the vast majority of sequences provide little to no measurable functionality. This leads to high-fitness latent encodings that are hidden within a dense point cloud of low-fitness latent encodings, leading to inefficiencies when searching latent space through small perturbations to z, Δ_{z}. As a result, the improvement process will spend much of its time traveling through dense regions of low-fitness candidates. Again, a strong link between changes in sequence information, now encoded in Δ_{z}, and changes its associated fitness Δy has yet to be established.

The present disclosure proposes to connect these two important factors through the use of an autoencoder model trained jointly on the fitness prediction task, thereby combining the described two-step process into one. This method adds to the autoencoder model architecture, comprised of an encoder f, decoder g, and a network h which is tasked with predicting fitness from the latent representation z. The final objective function of this set-up takes the form

$${L}_{\text{task}} = γ {L}_{\text{recon}} + α {L}_{\text{reg}} \qquad \text{Equation 4}$$

where .C_{recon} represents the reconstruction task and L_{reg} represents the fitness prediction task. Additionally, γ and a are scalar hyperparameters which weight their respective tasks. This model is referred to herein as a JT-AE.

$${z}^{\left(t + 1\right)} ← {z}^{\left(t\right)} - η ⋅ \left({∇}_{z} {L}_{\text{recon}} + {∇}_{z} {L}_{\text{reg}}\right) \qquad \text{Equation 5}$$

An important consequence of the joint training setup is that the latent code is updated during each training step with gradient signals from both sequence and fitness information. The resulting z encoding is thereby induced to resolve the two pieces of information. After model convergence, the latent space is endowed with a strong sequence-fitness association which is leveraged for latent space improvement.

$$L = \left|\left|g \left(f \left(x\right)\right) - x\right|\right| + \left|\left|h \left(f \left(x\right)\right) - y\right|\right| \qquad \text{Equation 6}$$

Furthermore, one can observe that in each update step, the encoder receives gradients from both the reconstruction loss and fitness prediction loss and is therefore directed to encode information about sequence and fitness in z. Indeed, when the dimensionality of z is set to some low value d « N, the latent encoder is forced to include only the most salient information about sequence and fitness and induces a greater connection between the two in z. Through the use of this training strategy, the connection between ∇_{z} and ∇_{y} is strengthened for downstream applications.

A pervasive challenge of calculating improvements within the latent space of deep learning models is moving far from the training data into regions where the model’s predictive performance deteriorates or is otherwise untrustworthy. Recent work has proposed techniques to define boundaries for model-based improvement methods, such as through a sequence mutation radius or by relying on model-derived likelihood values. In general, the gradients produced by a supervised network do not readily provide a stopping criterion nor any strong notion of bounds in regards to the range of values the network predicts. This can be further shown by training a network to predict from a 2-dimensional latent representation and overlaying the gradient directions onto the latent space. A unidirectional organization by the predicted attribute is the likely outcome, as shown in FIG. 3C.

Disclosed herein is a solution that addresses the challenge of moving away from training points in latent space by focusing on the function learned by the neural network. During training, the model intakes data in batches of size N randomly sampled from the training data. As output of the encoder module of ReLSO, these datapoints are encoded in a low-dimensional latent space. To keep latent embeddings close to the origin, a norm-based penalty is included in the encoding. This then allows for the generation of negative samples by randomly sampling high-norm points in latent space. M latent points are sampled with L2-norms greater than that of the largest L2-norm observed in the original N points A hyperparameter is used to scale the allowed difference between the max L2-norm of the training samples and the min L2-norm of the negative samples. In some embodiments, this hyperparameter may be assigned a value of 1.5. In other embodiments, the hyperparameter may have a value between 1.05 and 3, or 1.1 and 2, or 1.2 and 1.8, or 1.3 and 1.7, or 1.4 and 1.6, or about 1.5. The training samples and negative samples are then concatenated batchwise and passed through the fitness prediction network. In the calculation of the mean-squared regression loss, the predicted fitness values for the negative samples are compared to a preset value. In some embodiments of the methods disclosed herein, this value is set as the minimum observed fitness in the dataset. The fitness prediction loss term is now the following:

$$L = \frac{1}{N} {\sum }_{t = 1}^{n} {\left({y}_{i} - {\hat{y}}_{i}\right)}^{2} + \frac{1}{M} {\sum }_{t = 1}^{m} {\left({y}_{\text{neg}} - \text{min} \left(Y\right)\right)}^{2} \qquad \text{Equation 7}$$

While negative sampling effectively restricts the function learned by fitness prediction network h_{θ} to resemble a concave shape, the ability of neural networks to learn a wide variety of functions can still allow for complex, non-concave shaped solutions to persist. In the bottom rows of FIG. 3C, learning of a smooth fitness function is further encouraged using spectral norm regularized layers, labeled as “spectral.” This is compared to an un-regularized fully-connected layer, labeled as “base.”

Smoothness within the latent space encodings of the disclosed model play a major role in the disclosed approach, and smoothness is measured with a metric used in Castro, E., et al., 2020 IEEE International Conference on Big Data. (2020). A symmetric KNN graph was constructed from the latent codes Z = _{Zi,} _{Zj}, ... from a set of sequences such that z; and zⱼ are connected by an edge if either z; is within the K-nearest neighbors of _{Zj} or conversely, if zⱼ is within the K-nearest neighbors of _{Z}i. By constructing the graphs in this way, the disclosed metric is guaranteed to be scale-invariant. The KNN graph A is then used to construct the combinatorial graph Laplacian operator L = D - A from which the smoothness metric is calculated as

$${λ}_{y} = \frac{1}{N} {y}^{T} L y \qquad \text{Equation 8}$$

where y is the signal of interest and N corresponds to the number of datapoints used to construct the graph. The quadratic form of the graph Laplacian operator can be interpreted as taking the sum of squared differences along edges in the underlying graph such that the resulting sum is lower if the signal is smooth, i.e. with small differences between neighboring points.

### Datasets

Quantitative readouts of fitness landscapes have remained elusive until novel breakthroughs in high-throughput molecular biology, such as directed evolution and deep mutational scanning. Broadly speaking, these methods aim to introduce mutations in the sequence (or a set of interesting positions) in a systematic (saturation mutagenesis) or random (directed evolution) manner.

GIFFORD dataset: Enrichment data from directed evolution; in this experiment, Liu et al (Liu, G. et al. Bioinformatics (2020)) pitted a vast library (10¹⁰ unique mutants) of an antibody against a single target. This library was then pitted through three consecutive rounds of selection, washing, and amplification. Next-gen sequencing was used between rounds to identify which sequences were enriched. Here, the fitness was the log ratio of sequence enrichment between rounds of selection (i.e., how well the sequence performed relative to other members of the library).

GB1 dataset: Wu et al (Wu, N. et al., eLife (2016)) carried out a saturation mutagenesis study targeting four sites and generated all 20⁴ possible mutants to explore the local fitness landscape of GB1, an immunoglobulin-binding protein. This particular site is known to be an epistatic cluster. Fitness was measured by testing stability and binding affinity.

GFP dataset: Sarkisyan et al (Sarkisyan, K. S. et al., Nature (2016)) carried out random mutagenesis on a fluorescent protein (avGFP) to generate 51,175 unique protein coding sequences, with an averages of 3.7 mutations. Fitness was determined by measuring fluorescence of mutated constructs via a fluorescence-activated cell sorting (FACS) assay.

TAPE dataset: In addition to the datasets pulled from prior work, the TAPE benchmark datasets for fluorescence were used from Rao, R., et al., bioRxiv (2020)). Note that the train/test/validation splits were kept consistent so as to establish a fair comparison. The data here is the same as Sarkisyan et al., but is simply split by sequence distance.

### Improvement Methods

ReLSO is compared to two popular approaches for ML-based protein sequence improvement. These methods manipulate sequences directly and use a machine learning model to screen candidates, effectively treating model inference as a substitute for wet-lab characterization. First, the disclosed methods were compared against in-silico directed evolution, as described in Yang, K. K., et al., Nat. Methods (2019). Here a subset of residues from the protein sequence of interest were iteratively expanded and screened in-silico. The best amino acid for each position was then held constant while the next position was improved. Second, the disclosed method was compared against the Metropolis-Hastings Markov chain Monte Carlo approach used in Biswas, S., et al., Nat. Methods (2021) where protein sequences undergo random mutagenesis. All mutations with improved fitness are accepted into the next iteration and a few mutations with reduced fitness are also carried forward. In the disclosed comparisons, this approach is referred to as MCMC Seq and the directed evolution approach as DE.

The first set of gradient-free algorithms employ a local search where a small perturbation in the latent encoding is added zₜ₊₁ = zₜ + _{E}, where t is the step index and zₜ₊₁ is accepted with a probability equal to min

$$\left(1 , {e}^{\frac{{y}_{t + 1} - {y}_{t}}{k T}}\right) .$$

The second group of gradient-free improvement algorithms use a nearest-neighbors search and either move in the direction of the most fit neighbor (hill climbing) or choose uniformly from the set (D = zⱼₗh(zⱼ) = yⱼ > _{Yi} (stochastic hill climbing). Since the fitness prediction head is trained directly from the latent encoding, the gradients of this network are accessible and one can perform gradient ascent. The effect of cycling candidates back through the model was also examined as denoted in Equation 2. Two gradient-free methods were examined which operate in sequence space. One such method is a form of in-silico directed evolution where positions are independently and sequentially improved. The second improvement approach mirrors that of the Metropolis-Hastings Monte Carlo search approach used in latent space with the exception that the perturbation step is replaced with a mutation step.

Next a set of algorithms is considered that operate in the latent space of generative models. These methods still treat the prediction network as a black box, unable to access gradients, but manipulate sequences using their latent encodings. First, a simple hill-climbing algorithm (HC) is examined which takes a greedy search through latent space. A stochastic variant of hill climbing was also evaluated, which should better avoid local minima. Here the algorithm samples zₜ₊₁ uniformly from {z | hₑ (z + _{E}) > hₑ (zₜ), _{E} - J\f(p, k) }, where k is a parameter. This variation of hill climbing is referred to as stochastic hill climbing (SHC). Furthermore, an MCMC scheme similar to the previously described approach of Biswas, S., et al., Nat. Methods (2021) was used, however in the present methods the improvement calculation was performed in latent space. A small perturbation was applied in the latent encoding zₜ₊₁ = zₜ + _{E}, where t is the step index and zₜ₊₁ is kept according to a probabilistic acceptance step. Lastly, the approach of Brookes, D. H. (2018); and Brookes, D., Proceedings of the 36th International Conference on Machine Learning (2019) were considered, where an adaptive sampling procedure is done to generate improved sequences. These approaches are referred to herein as DbAS and CbAS.

The performance of a latent space gradient ascent improvement method was examined. Here the ability to extract gradient directions provided by the j ointly-trained fitness prediction head of the model h_{θ} was examined. These directions allow for latent space traversal to areas of latent space associated with higher fitness sequences. This approach was first examined through a jointly-trained autoencoder without the aforementioned regularizations and this approach is denoted as JTAE — GA. Next, the performance of gradient ascent using the interpolation sampling and negative sampling regularizations of ReLSO was examined, and referred to herein as ReLSO — GA.

The disclosed method was also compared to the DbAS and CbAS methods introduced in Brookes, D. H. (2018); and Brookes, D., Proceedings of the 36th International Conference on Machine Learning (2019), respectively. In this approach, the fitness prediction model is treated as a black-box “oracle” which maps from design space to a distribution over properties of interest. Similar to the disclosed approach, the authors of these methods consider the pathologies inherent to relying on deep learning models to improve protein sequences. To address this, DbAS and CbAS use a model-based adaptive sampling technique which draws from the latent space of a generative model. While DbAS assumes an unbiased oracle, CbAS conditions the sampling procedure on the predictions of a set of oracle models. In the present disclosure, an implementation sourced from a publicly available GitHub repository was used (https://github.com/dhbrookes/CbAS). To ensure representative performance, DbAS and CbAs were first evaluated on the datasets using a grid search over several values of q ([0.5, 0.6, 0.7, 0.8]) and numbers of epochs used in training ([1,5,10,15]).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

### REFERENCES

The following publications are incorporated herein by reference.

Tiessen, A., Perez-Rodriguez, P. & Delaye-Arredondo, L. J. Mathematical modeling and comparison of protein size distribution in different plant, animal, fungal and microbial species reveals a negative correlation between protein size and protein number, thus providing insight into the evolution of proteomes. BMC Res. Notes 5, 85 (2012).

Starr, T. N. & Thornton, J. W. Epistasis in protein evolution. Protein Sci. 25, 1204-1218 (2016).

Romero, P. A. & Arnold, F. H. Exploring protein fitness landscapes by directed evolution. Nat. Rev. Mol. Cell Biol. 10, 866-876 (2009).

Chen, K. & Arnold, F. H. Engineering new catalytic activities in enzymes. Nat. Catal. 3, 203-213 (2020).

Arnold, F. H. Design by directed evolution. Acc. Chem. Res. 31, 125-131 (1998).

Rohl, C. A., Strauss, C. E. M., Misura, K. M. S. & Baker, D. Protein structure prediction using Rosetta. Methods Enzymol. 383, 66-93 (2004).

Norn, C. et al. Protein sequence design by conformational landscape optimization. Proc. Natl Acad. Sci. USA 118, e2017228118 (2021).

Brookes, D. H. & Listgarten, J. Design by adaptive sampling. Preprint at https://arxiv.org/abs/1810.03714 (2018).

Brookes, D., Park, H. & Listgarten, J. Conditioning by adaptive sampling for robust design. Proceedings of the 36th International Conference on Machine Learning 97, 773-782 (2019).

Yang, K. K., Wu, Z. & Arnold, F. H. Machine-learning-guided directed evolution for protein engineering. Nat. Methods 16, 687-694 (2019).

Biswas, S., Khimulya, G., Alley, E. C., Esvelt, K. M. & Church, G. M. Low-n protein engineering with data-efficient deep learning. Nat. Methods 18, 389-396 (2021).

Linder, J. & Seelig, G. Fast differentiable DNA and protein sequence optimization for molecular design. Preprint at https://arxiv.org/abs/2005.11275 (2020).

Angermueller, C. et al. Model-based reinforcement learning for biological sequence design. In International Conference on Learning Representations (2019).

Alley, E. C., Khimulya, G., Biswas, S., AlQuraishi, M. & Church, G. M. Unified rational protein engineering with sequence-based deep representation learning. Nat. Methods 16, 1315-1322 (2019).

Liu, G. et al. Antibody complementarity determining region design using high-capacity machine learning. Bioinformatics 36, 2126-2133 (2020).

Jumper, J. et al. Highly accurate protein structure prediction with AlphaFold. Nature 596, 583-589 (2021).

Rao, R. et al. Evaluating protein transfer learning with TAPE. Adv. Neural Inf. Process. Syst. 32, 9689-9701 (2019).

Rao, R., Ovchinnikov, S., Meier, J., Rives, A. & Sercu, T. Transformer protein language models are unsupervised structure learners. Preprint at bioRxiv https://doi.org/10.1101/2020.12.15.422761 (2020).

Rives, A. et al. Biological structure and function emerge from scaling unsupervised learning to 250 million protein sequences. Proc. Natl Acad. Sci. USA 118, e2016239118 (2021).

Vig, J. et al. BERTology meets biology: interpreting attention in protein language models. Preprint at https://arxiv.org/abs/2006.15222 (2020).

Hochreiter, S. The vanishing gradient problem during learning recurrent neural nets and problem solutions. Int. J. Uncertain. Fuzziness Knowl.-Based Syst. 6, 107-116 (1998).

Detlefsen, N. S., Hauberg, S. & Boomsma, W. Learning meaningful representations of protein sequences. Nat. Commun. 13, 1914 (2022).

Gomez-Bombarelli, R. et al. Automatic chemical design using a data-driven continuous representation of molecules. ACS Cent. Sci. 4, 268-276 (2018).

Castro, E., Benz, A., Tong, A., Wolf, G. & Krishnaswamy, S. Uncovering the folding landscape of RNA secondary structure using deep graph embeddings. 2020 IEEE International Conference on Big Data. 4519-4528 (2020).

Sarkisyan, K. S. et al. Local fitness landscape of the green fluorescent protein. Nature 533, 397-401 (2016).

Rodrigues, C. H., Pires, D. E. & Ascher, D. B. Dynamut2: assessing changes in stability and flexibility upon single and multiple point missense mutations. Protein Sci. 30, 60-69 (2021).

Vaswani, A. et al. Attention is all you need. Adv. Neural Inf. Process. Syst. 30 (2017).

Yoshida, Y. & Miyato, T. Spectral norm regularization for improving the generalizability of deep learning. Preprint at https://arxiv.org/abs/1705.10941 (2017).

Mistry, J. et al. Pfam: the protein families database in 2021. Nucleic Acids Res. 49, D412-D419 (2021).

Wu, N. C., Dai, L., Olson, C. A., Lloyd-Smith, J. O. & Sun, R. Adaptation in protein fitness landscapes is facilitated by indirect paths. eLife 5, e16965 (2016).

Moon, K. R. et al. Phate: a dimensionality reduction method for visualizing trajectory structures in high-dimensional biological data. BioRxiv 120378 (2017).

We claim:

1. A system for identifying biomolecules with a desired property, comprising:
a non-transitory computer-readable medium with instructions stored thereon, which when executed by a processor perform steps comprising:
collecting a quantity of biomolecular data;
transforming the biomolecular data from a sequence space to a latent space representation of the biomolecular data;
compressing the latent space representation of the biomolecular data to a coarse representation using a pooling mechanism;
compressing the coarse representation of the biomolecular data to a low-dimensional representation of the biomolecular data using an informational bottleneck;
organizing the data in the low-dimensional representation of the biomolecular data according to a fitness factor;
choosing a first point from within the low-dimensional representation of the biomolecular data;
calculating a gradient of the fitness factor at the first point in the low-dimensional representation of the biomolecular data;
selecting a second point in the low-dimensional representation of the biomolecular data in a direction indicated by the gradient to have a higher fitness factor than the first point, setting the second point as the first point, then repeating the gradient calculating step until the fitness factor reaches a convergence point or threshold value; and
transforming the selected point from within the low-dimensional representation of the biomolecular data back to the sequence space to identify an improved candidate sequence.

2. The system of claim 1, wherein the pooling mechanism is an attention-based pooling mechanism.

3. The system of claim 1, wherein the pooling mechanism is a mean or max pooling mechanism.

4. The system of claim 1, wherein the pooling mechanism is a recurrent pooling mechanism.

5. The system of claim 1, wherein the informational bottleneck is an autoencoder-type bottleneck.

6. The system of claim 1, the instructions further comprising the step of adding negative samples to the latent space representation of the biomolecular data.

7. The system of claim 6, wherein the negative samples have a fitness value less than or equal to the minimum fitness value calculated in the latent space.

8. The system of claim 1, wherein the biomolecular data comprises sequencing data of at least one lead biomolecule.

9. The system of claim 1, wherein the instructions comprise transforming the biomolecular data to a latent space representation of the biomolecular data with a transformer module having at least eight layers with four heads per layer.

10. A method of identifying biomolecules with a desired property, comprising:
collecting a quantity of biomolecular data;
transforming the biomolecular data from a sequence space to a latent space representation of the biomolecular data;
compressing the latent space representation of the biomolecular data to a coarse representation using a pooling mechanism;
compressing the coarse representation of the biomolecular data to a low-dimensional representation of the biomolecular data using an informational bottleneck;
organizing the data in the low-dimensional representation of the biomolecular data according to a fitness factor;
choosing a first point from within the low-dimensional representation of the biomolecular data;
calculating a gradient of the fitness factor at the first point in the low-dimensional representation of the biomolecular data;
selecting a second point in the low-dimensional representation of the biomolecular data in a direction indicated by the gradient to have a higher fitness factor than the first point, setting the second point as the first point, then repeating the gradient calculating step until the fitness factor reaches a convergence point or threshold value; and
transforming the selected point from within the low-dimensional representation of the biomolecular data back to the sequence space to identify an improved candidate sequence.

11. The method of claim 10, wherein the pooling mechanism is an attention-based pooling mechanism.

12. The system of claim 10, wherein the pooling mechanism is a mean or max pooling mechanism.

13. The method of claim 10, wherein the pooling mechanism is a recurrent pooling mechanism.

14. The method of claim 10, wherein the informational bottleneck is an autoencoder-type bottleneck.

15. The method of claim 10, the instructions further comprising the step of adding negative samples to the latent space representation of the biomolecular data.

16. The method of claim 15, wherein the negative samples have a fitness value have a fitness value less than or equal to the minimum fitness value calculated in the latent space.

17. The method of claim 10, wherein the biomolecular data comprises sequencing data of at least one lead biomolecule.

18. The method of claim 10, wherein the instructions comprise transforming the biomolecular data to a latent space representation of the biomolecular data with a transformer module having at least eight layers with four heads per layer.

19. The method of claim 10, further comprising producing a protein with the improved candidate sequence.
